(19) United States
(12) Patent Application Publication
(10) Pub. No.: US 2023/0058519 A1
(43) Pub. Date: Feb. 23, 2023

(54) Cell Expressing Immune Modulatory Molecules and System for Expressing Immune Modulatory Molecules

(71) Applicant: Shanghai Cell Therapy Group Company Co., Ltd., Shanghai (CN)

(72) Inventor: Qijun Qian, Shanghai (CN)

(21) Appl. No.: 17/758,128

(22) PCT Filed: Dec. 28, 2020
(86) PCT No.: PCT/IB2020/001079

§ 371 Date: Jun. 28, 2022

Related U.S. Application Data
(60) Provisional application No. 62/954,448, filed on Dec. 28, 2019.

(51) Int. Cl. C12N 5/0783; C07K 16/28; C07K 16/30; C12N 15/63

(52) U.S. Cl. CPC C12N 5/0638; C07K 16/2818; C07K 16/2827; C07K 16/30; C12N 15/63; C07K 2317/31; C07K 2317/24; C07K 2317/569

(57) ABSTRACT

Presently disclosed are immune cells (i.e., the Baize Super Cells) that have been engineered to express and incorporate an immune cell activator polypeptide comprising an extracellular label domain into their cell surface membrane. Also disclosed are immune cells that have been engineered to secrete one or more polypeptide effector molecules, as well as immune cells engineered to express both molecules. Nucleic acid vectors for expressing these molecules in immune cells are disclosed. A bispecific polypeptide that can be used to specifically bind an immune cell expressing an immune cell activator polypeptide to another cell is also disclosed. A system including both the immune cells and various bispecific polypeptides that bind to different cell surface proteins on the same or different cell targets, which can be used to proliferate the immune cells in vivo and treat various kinds of tumors, for example, is also disclosed.

### FIELD

The subject matter disclosed herein relates to cells (Baize Super Cells) that express immune system modulatory proteins or other effector polypeptides, chimeric immune cell activator polypeptides (ICAPs) and to systems that are used to control the expression of such proteins and polypeptides in those cells. Such systems can include polypeptides that have bispecific binding activities and so can activate cells harboring vectors for expressing immune system modulatory proteins or other effective polypeptides upon also binding to a polypeptide target domain.

### BACKGROUND

Chimeric Antigen Receptor (CAR) bearing T cells (CAR-T cells) are being developed as an immunotherapeutic mode of cancer treatment. In general, a CAR includes an extracellular domain that binds an activating ligand, a transmembrane domain that participates in forming an immune synapse with a “target” cell and an intracellular domain that responds to binding of the extracellular domain by activating T-cell associated transcriptional responses.

Present CAR-T cell based therapies are not effective against tumors with heterogeneous TAA (tumor associated antigen) expression or emerging antigen loss variants due to a single TAA recognizing extracellular domain in the CAR.

Present CAR-T cell based therapies rely on in vitro proliferation of CAR-T cells before patient treatment.

Furthermore, no easy method is available for in vivo monitoring of CAR-T cell distribution and fate.

Still further CAR-T cells continually and uncontrollably proliferate and activate in response to antigen, potentially causing fatal on-target off-tumor toxicity, cytokine release syndrome, or neurotoxicity in the absence of any a method of control of the activated CAR-T cell activity or method for elimination of undesired CAR-T cells.

Most of CAR extracellular antigen-recognizing domains are scFv proteins and it has become evident that two scFv domains can form a non-covalently linked dimer, such as by domain swapping. This type of interaction between neighboring scFv domains strongly enhances the tonic signaling in CAR-T cells, which leads uncontrollable activity.

### SUMMARY OF THE DISCLOSURE

Presently disclosed are immune cells that have been engineered to express and incorporate an immune cell activator polypeptide (ICAP) into their cell surface membrane. Also disclosed are immune cells that have been engineered to secrete one or more polypeptide effector molecules, as well as immune cells engineered to express both molecules.

Thus, in one aspect of the disclosure there is provided an immune cell that comprises a (or a first) nucleic acid vector comprising:
(a) a promoter region effective for transcription in an immune cell;
(b) a polynucleotide encoding an amino acid sequence of the immune cell activator polypeptide; and
(c) a terminator region effective for ending transcription in an immune cell. Such an immune cell can be one that further comprises a second nucleic acid vector comprising
(d) a promoter region effective for transcription in an immune cell;
(e) a polynucleotide encoding an amino acid sequence of one or more secreted polypeptide effector molecules; and
(f) a terminator region effective for ending transcription in an immune cell.

Alternatively, the engineered immune cell can be one wherein the first nucleic acid vector further comprises a polynucleotide encoding an amino acid sequence of one or more secreted polypeptide effector molecules.

Another aspect of the disclosure resides in an immune cell activator polypeptide comprising:
(a) a label domain;
(b) a transmembrane domain; and
(c) a signal transduction domain.

A further aspect of the disclosure is a nucleic acid vector comprising:
(a) a promoter region effective for transcription in an immune cell;
(b) a polynucleotide encoding an amino acid sequence of an immune cell activator polypeptide; and
(c) a terminator region effective for ending transcription in an immune cell.

Another aspect of the disclosure is a nucleic acid vector comprising:
(a) a promoter region effective for transcription in an immune cell;
(b) a polynucleotide encoding an amino acid sequence of one or more secreted polypeptide effector molecules.
(c) a terminator region effective for ending transcription in an immune cell.

Yet another aspect of the disclosure is a bispecific polypeptide that is a nanobody targeting and control polypeptide (VHH-TCP) comprising:
(a) a label-binding domain (L-bd) comprising a single chain polypeptide domain that specifically binds to a label domain of an immune cell activator polypeptide; and
(b) a cell surface protein-binding domain (CSP-bd) comprising a single chain polypeptide domain that specifically binds to a cell surface receptor of a cell.

The disclosure also describes a kit for in situ production of one or more polypeptide effector molecules proximal to a target cell comprising:

I. an immune cell that comprises a nucleic acid vector comprising
(a) a promoter region effective for transcription in an immune cell;
(b) a polynucleotide encoding an amino acid sequence of the immune cell activator polypeptide comprising a signal transduction domain, a transmembrane domain, and a label domain; and
(c) a terminator region effective for ending transcription in an immune cell; and

a second nucleic acid vector comprising
(a) a promoter region effective for transcription in an immune cell;
(b) a polynucleotide encoding an amino acid sequence of one or more secreted polypeptide effector molecules;
(c) a terminator region effective for ending transcription in an immune cell; and

II. a bispecific polypeptide comprising:
(a) a label-binding domain (L-bd) comprising a single chain polypeptide domain that specifically binds to a label domain of an immune cell activator polypeptide; and
(b) a cell surface protein-binding domain (CSP-bd) comprising a single chain polypeptide domain that specifically binds to a cell surface receptor of a cell.

Alternatively, the engineered immune cell can be one wherein the first nucleic acid vector further comprises a polynucleotide encoding an amino acid sequence of a secreted effector polypeptide. In such an embodiment, the secreted effector polynucleotide can be encoded in a second expression cassette. Such a kit further comprises a bispecific polypeptide that is a nanobody targeting and control polypeptide (VHH-TCP) comprising:
(a) a label-binding domain (L-bd) comprising a single chain polypeptide domain that specifically binds to a label domain of an immune cell activator polypeptide; and
(b) a cell surface protein-binding domain (CSP-bd) comprising a single chain polypeptide domain that binds to a cell surface receptor of a cell.

The disclosure also provides a method for modulating the immune system environment in the locality of a tumor cell in a subject comprising:
(a) administering to a subject an effective amount of an engineered immune cell that comprises a first nucleic acid vector comprising:
   (i) a promoter region effective for transcription in an immune cell;
   (ii) a polynucleotide encoding an amino acid sequence of the immune cell activator polypeptide; and
   (iii) a terminator region effective for ending transcription in an immune cell.
and that further comprises a second nucleic acid vector comprising
   (i) a promoter region effective for transcription in an immune cell;
   (ii) a polynucleotide encoding an amino acid sequence of one or more secreted polypeptide effector molecules; and
   (iii) a terminator region effective for ending transcription in an immune cell;
(b) concurrently or sequentially administering to the subject an effective amount of a first bispecific polypeptide comprising:
   (i) a label-binding domain (L-bd) comprising a single chain polypeptide domain that specifically binds to a label domain of an immune cell activator polypeptide; and
   (ii) a cell surface protein-binding domain (CSP-bd) comprising a single chain polypeptide domain that specifically binds to a cell surface protein of a lymphocyte;
(c) administering to the subject an effective amount of a second bispecific polypeptide comprising:
   (i) a label-binding domain (L-bd) comprising a single chain polypeptide domain that specifically binds to a label domain of an immune cell activator polypeptide; and
   (ii) a cell surface protein-binding domain (CSP-bd) comprising a single chain polypeptide domain that specifically binds that specifically binds to a cell surface protein of the tumor cell.

A step of measuring the amount of engineered immune cells in the subject can be performed between steps b and c.

A method for modulating the immune system environment in the locality of a tumor cell in a subject can alternatively comprise:
(a) proliferating a transformed T-cell of the subject in vitro, wherein the T-cell comprises a first nucleic acid vector that comprises a nucleic acid vector comprising:
   (i) a promoter region effective for transcription in an immune cell;
   (ii) a polynucleotide encoding an amino acid sequence of an immune cell activator polypeptide; and
   (iii) a terminator region effective for ending transcription in an immune cell;
and comprises a second nucleic acid vector that comprises
   (i) a promoter region effective for transcription in an immune cell;
   (ii) a polynucleotide encoding an amino acid sequence of one or more secreted polypeptide effector molecules; and
   (iii) a terminator region effective for ending transcription in an immune cell;
to obtain proliferated T-cells; and administering the proliferated T-cells into the subject; and
(b) administering to the subject an amount effective to activate the proliferated T-cells to express the secreted polypeptide effector molecule of a VHH-TCP that comprises a L-bd of a determined amino acid sequence that specifically binds to the label domain expressed by the proliferated T-cells and a CSP-bd that specifically binds to a cell surface protein of the tumor cell.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements and in which:

FIG. 1 depicts an exemplary effector cell as described herein, such as a “Baize Super Cell,” showing expression of an immune cell activator polypeptide and secreting an immunomodulatory effector molecule; an anti-PD1-VHH nanobody is illustrated.

FIG. 2 depicts a nanobody-targeting and control polypeptide (VHH-TCP); domains binding to the label domain of an immune cell activator protein (label-VHH) and to a cell surface protein of a cell, in this instance the CD19 ligand of a B cell (CD19-VHH), are illustrated. Additional domains for activating Fc-mediated immune responses (hFc-VHH), binding of a FITC fluorophore (FITC-VHH) and binding to serum albumin (albumin-VHH) are also shown.

FIGS. 3A and 3B depict maps of exemplary expression vectors for expressing an ICAP and an effector protein in an immune cell type host cell. In FIG. 3A, an effector protein anti-PD-1-VHH-Fc (EQ) is expressed from the structural gene in the expression construct within the vector pS338B-1182-Fc (EQ). In FIG. 3B, an ICAP having a label polypeptide domain, a CD8 hinge domain, a CD28 transmembrane (TM) domain and a CD28 intracellular signaling domain and a CD3z domain is expressed from the structural gene in the expression construct pNB338B-ICAPs-VHH.

FIG. 4A shows flow binding affinity of M 2339(VHH) towards mesothelin (MSLN). FIG. 4B shows flow binding affinity of B029(VHH) towards BCMA. FIG. 4C shows flow binding affinity of E454(VHH) towards EGFR.

FIG. 5 shows the binding kinetics of M2339 VHH-6 his binding to different mesothelin ECD domains determined by Surface Plasmon Resonance (SPR).

FIG. 6 presents schematic diagrams of various M-ICAP (origin from mesothelin II+III region) vectors. 19R73 is the version of the canonical CD19CAR-T (positive control), and others are M-ICAP vectors. The intracellular regions of these vectors are same, which all contain 4-BB and CD3ζ, but the extracellular regions are different. M-ICAP does not contain 6-His tags. His-1/2-M-ICAP: 6-His tags are located at the N-end or C-end of M-ICAP. SP3-His-M-ICAP and SP5His-M-ICAP: signal peptides are SP3 or SP5, which are selected from human protein database. SP3 (signal peptide 3): MKHLWFFLLLVAAPRWVLS (SEQ ID NO:1); SP5 (signal peptide 5): MTRLTVLALLAGLLASSRA (SEQ ID NO:2);

FIG. 7 shows the FACS results of M-ICAP vectors transfected into 293T cell. FIG. 7A shows the positive ratio of different M-ICAP vectors transfected into 293T cell. FIG. 7B shows the dot plot of FACS results.

FIG. 8 illustrates M-ICAP expression and M-ICAP-T cell construction.

FIG. 8A is a schematic diagram of M-ICAP-T cell construction. FIG. 8B provides schematic diagrams of M-ICAP, SP3-M-ICAP, and SP5-M-ICAP expression vectors. FIGS. 8C and 8D present data of Positive ratio of M-ICAP, SP3-M-ICAP, SP5-M-ICAP T cells 8 and 13 days after transfection (FIG. 8C: activated by M2339+antiCD28 or antiHis+antiCD28 respectively; FIG. 8D: activated by M2339+antiCD28). Abbreviations: M-ICAP—peptide origin from mesothelin II+III; SP—mesothelin—endogenous signal peptide of mesothelin; SP3 (signal peptide 3): MKHLWFFLLLVAAPRWVLS (SEQ ID NO:1); SP5 (signal peptide 5): MTRLTVLALLAGLLASSRA (SEQ ID NO:2); M2339, antiM-ICAP—VHH-Fc clone M2339; antiCD28—anti-CD28 mAb; antiHis—anti-His mAb.

FIG. 9 shows a representative preparation and quality verification of ICAP-T cells. FIG. 9A provides schematic diagrams of M-ICAP, M-ICAP-28, M-ICAP-28BB expression vectors. FIG. 9B shows a comparison of ICAP-T amplification (from peripheral blood monocytes, PBMC) obtained by different TCP or antibody activation. FIG. 9C shows expansion during preparation of ICAP-T cells from PBMC. FIG. 9D shows the ICAP-positive proportion of ICAP-T cell product. FIG. 9E shows the CD4/CD8 positive proportion in CD3 positive cells of the ICAP-T-cell product. FIG. 9F shows the Tem/Tcm positive proportion in Tm cells of the ICAP-T cell product.

FIG. 10 shows binding affinity of BCMA-TCPs measured by FACS. FIG. 10A shows the FACS binding curve of three BCMA-TCPs to cells of an MSLN over-expression cell line.

FIG. 10B shows the FACS binding curve of three BCMA-TCPs to cells of a BCMA over-expression cell line.

FIG. 11 shows the plasma stability of anti-BCMA TCPs.

FIG. 12 shows the binding affinity of TCP011-P to two different cell types measured by FACS. FIG. 12A shows the FACS binding curve of TCP011-P to cells of an CD19 over-expression cell line. FIG. 12B shows the FACS binding curve of TCP011-P to cells of a MSLN over-expression cell line.

FIG. 13 shows binding affinity of TCP021-P to two different cell types measured by FACS. FIG. 13A shows the FACS binding curve of TCP021-P to EGFR over-expressing cells. FIG. 13B shows the FACS binding curve of TCP021-P to MSLN over-expressing cells.

FIG. 14 shows the in vitro amplification of M-ICAP-T with TCP towards target cells. FIGS. 14A and 14B show the count of T/Daudi cells after 4 days co-culture of M-ICAP transfected T and Daudi cells with TCP. FIGS. 14C and 14D show the count of T/Daudi cells after 4 days co-culture of M-ICAP transfected T and mitomycin C (MMC) treated Daudi cells with TCP.

FIG. 15 shows TCP-dose-dependent cytotoxicity effect of M-ICAP-T towards RPMI-8226 cells. FIG. 15A shows a schematic diagram of the suspension cell cytolysis assay.

FIG. 15B shows the dose-dependent cytolysis release ratio of M-ICAP-T combined TCP001-C to RPMI-8226 cells in three different E:T ratio. FIGS. 15C-15E show the cytolysis analysis curve of different E:T ratios.

FIG. 16 shows a comparison of cytotoxicity and IFNγ secretion of ICAP/CAR-T combined with different TCPs towards RPMI-8226/L363 cells. FIGS. 16A and 16B show the comparison of cytotoxic effect of ICAP/CAR-T cells combined with different TCPs to L363 cells at concentration of 0.5 (A) or 0.2 (B) ug/ml. FIGS. 16C and 16D show the IFNγ secretion of ICAP/CAR-T combined with different TCPs towards RPMI-8226(C) or L363(D) cells.

FIG. 17 shows the cytolysis effect of ICAP combined with TCP (binding EGFR) on FaDu/SK-OV3 cells. FIG. 17A shows the cytolysis of FaDu cells by ICAP/CAR-T cells combined with different TCPs. FIG. 17B shows the cytolysis of SK-OV3 cells by ICAP/CAR-T cells combined with different TCPs.

FIG. 18 shows IFN-γ release and cytolysis effect of ICAP-T cells with TCPs towards Daudi cells. FIG. 18A shows the IFN-γ release of ICAP/CAR-T cells with different TCPs on Daudi cells. FIG. 18B shows the cytolysis of Daudi cells by ICAP/CAR-T cells combined with different TCPs.

FIG. 19 shows M-ICAP-T has the ability to secrete antibodies, and the positive rate is not affected. FIG. 19A shows the positive rate comparison of secretory M-ICAP-T cells. Human naive T cells were simultaneously transfected with M3 CAR and secreted antibody's plasmids, such as anti-PD-1, anti-TGFβ, and anti-PD-L1. After 13 days, little difference existed among the four experimental groups, which is about 60-70% positive ratio. Anti-PD-1, anti-TGFβ, and anti-PD-L1 antibodies were also well secreted from M-ICAP-T cells. These data show that the type and level of antibody secretion have little effect on the positive conversion of M-ICAP-T cells. Either VHH or scFv can be well secreted from M-ICAP-T cells and detected by ELISA.

FIG. 20 shows that anti-PD-1-M-ICAP-T can secret anti-PD-1 VHH to block the surface PD-1 protein.

Cells were stimulated with 5 ug/ml M2339-IgG4 or IgG4 control for 48 hours. Only surface PD1 detection of anti-PD-1 VHH M-ICAP-T cells group is blocked by commercial PD-1 mAbs.

FIG. 21 shows anti-TGFβ scFv secreted by M-ICAP-T binds to TGFβRII. TGFβ ligands bind to TGFβRII and stimulate luciferase signals. Anti-TGFβ scFvs secreted by M-ICAP-T cells can also bind to TGFβRII on 293T cells and block luciferase reporter expression. CAR-T-10C, 10B and 01A are anti-TGFβ M-ICAP-T cells which were prepared from different donors.

FIG. 22 shows body weight change of L363-PDL1 orthotopic tumors in NPSG mice in the in vivo efficacy test of M-ICAP-T cells combined with TCP001-C.

FIG. 23 shows tumor volume change of L363-PDL1 orthotopic tumors in NPSG mice in the in vivo efficacy test of M-ICAP-T cells combined with TCP001-C.

FIG. 24 shows analysis of anti-PD-1VHH and TCP001-C concentration in whole blood of mice. FIG. 24A shows the analysis of serum anti-PD-1 VHH level. FIG. 24B shows the analysis of serum TCP001-C level. Abbreviations: D15-24 h, tail-vein bleed at D15, 24 h after injection of TCP001-C at Day14; D22-48 h, tail-vein bleed at D22, 48 h after injection of TCP001-C at Day 20.

The promoter in each of the vectors shown is an EF1a promoter and a SV40 polyadenylation signal is used for transcription termination in both vectors. The expression constructs both include 5′ and 3′ ITR sequences.

FIG. 25 shows binding of anti-MSLN-1444 VHH (1444 (VHH)) on HEK293T-MSLN cells analyzed by FACS.

FIG. 26 shows expression of the fusion polypeptide BCMA ICAP BCMAmut1 with anti-MSLN-1444 VHH.

FIG. 27 shows SPR Kinetics of BCMA ICAP BCMAmut1 binding to different anti-BCMA VHHs.

FIG. 28 shows in vitro activation and amplification of BCMAmut1-MSLN-1444 CAR-T. FIG. 28A shows the schematic presentation of BCMAmut1-MSLN-1444 vector; FIG. 28B., amplification of BCMAmut1-MSLN-1444 CAR-T stimulated with anti-BCMAmut1 VHH 36# or anti-MSLN in donor 1; and FIG. 28C, amplification of BCMAmut1-MSLN-1444 CAR T stimulated with anti-BCMAmut1 VHH 36# or anti-MSLN in donor 2.

FIG. 29 shows dot plot of FACS results of amplification of BCMAmut1-MSLN-1444 CAR-T stimulated with anti-BCMAmut1 VHH 36# or anti-MSLN in 2 donors.

FIG. 30 shows anti-BCMAmuc1 VHH 36# specific activation and amplification of MSLN-1444 CAR-T. FIG. 30A shows a schematic presentation of MSLN-1444 vector. FIG. 30B shows amplification of MSLN-1444 CART stimulated with anti-BCMAmuc1 VHH 36# or antigen MSLN in donor 1. FIG. 30C shows amplification of MSLN-1444 CAR T stimulated with anti-BCMAmuc1 VHH 36# or anti-MSLN in donor 2.

### MODES OF CARRYING OUT THE INVENTION

Chimeric antigen receptor T cell (CAR-T) treatment technology is a field of immune cell therapy for cancer. CAR-T technology uses genetic engineering technology to splice, e.g. an antibody variable region gene sequence including at least one portion of the gene encoding a CDR portion of the antibody, with the intracellular region of a T lymphocyte immune receptor, and then to introduce the splice construct into a T cell by retrovirus or lentiviral vector, transposon or transfection. The expression cassette or a mRNA is transduced into lymphocytes and expresses the fusion protein on the cell surface, enabling T lymphocytes to recognize specific antigens in a non-MHC-restricted manner, enhancing their ability to recognize and kill tumors.

The structure of a Chimeric Antigen Receptor (CAR) was proposed by the Eshhar research team in Israel in 1989. Since then, it has been confirmed that T cells displaying a CAR-structured cell surface protein have a good effect in tumor immunotherapy.

The first generation of CAR receptors contained a single-chain variable fragment (scFv), and the intracellular activation signal was transmitted by a CD3ζ (CD3z) signal chain. However, first-generation CAR receptors lack a domain to provide a T cell costimulatory signal, which leads to the CAR-T cells only exerting transient effects, short survival time of the cells in the body and less secretion of cytokines. The second generation of CAR receptors introduces the intracellular domain of costimulatory signaling molecules, including, for example, CD28, CD134/OX40, CD137/4-1BB, lymphocyte-specific protein tyrosine kinase (LCK), inducible T-cell co-stimulator (ICOS), DNAX-activation protein 10 (DAP10) and other domains to enhance T cell proliferation and cytokine secretion. IL-2, IFN-γ and GM-CSF production increase, thereby breaking the immunosuppression of the tumor microenvironment, for example AICD (activation induced cell death (AICD)).

Third-generation CAR receptors recombine a secondary co-stimulatory molecule such as 4-1BB between the co-stimulatory structure CD28 and an ITAM signal chain, thus producing a triple-signal CAR receptor.

Engineered CAR-T cells have better effector function and survival time in vivo. Presently, the CAR structure commonly used in therapies is a second-generation CAR receptor, and its structure can be divided into the following four parts: an antibody single-chain variable region (scFv), a hinge region, a transmembrane region, and an intracellular stimulation signaling polypeptide. The CAR hinge region structure contributes to forming the correct conformation and forming a dimer. The length of the hinge region and the amino acid sequence characteristics contribute to determining the spatial conformation of the CAR and also affect the ability of the CAR to bind to tumor cell surface antigens.

Malignant lymphoma is divided into two categories: Hodgkin's lymphoma (HL) and non-Hodgkin's lymphoma (NHL). Hodgkin's lymphoma accounts for 10%-15% of lymphoma, while Non-Hodgkin's lymphoma is the fastest-growing malignancy in patients with onset. According to WHO statistics, there are currently about 350,000 new NHL patients in the world each year, and the death toll exceeds 200,000. B-cell lymphoma can be seen in both of Hodgkin's lymphoma and non-Hodgkin's lymphoma. Currently, clinical treatments for lymphoma include cytotoxic drugs such as glucocorticoids and alkylating agents, and targeted drugs based on specific molecular targets (such as rituximab, etc.), wherein combination chemotherapy based on targeted drugs significantly improves responses, clinical remission rate and cure rate of patients. However, there are still a large number of patients with lymphoma who are not sensitive to or have poor efficacy and are “real” refractory patients. Some new treatments (such as cellular immunotherapy) have relieved and prolonged survival in patients with partially relapsed or refractory lymphoma. There are many types of CAR-Ts currently being developed for hematological malignancies, including therapies using anti-CD19, anti-CD20, anti-Kappa light chain, anti-CD22, anti-CD23, anti-CD30, anti-CD70 and other antibodies to construct CAR-modified T cells. Antitumor studies have been conducted and in these anti-CD19 and anti-CD20 monoclonal antibodies were the most commonly used antibodies.

Choosing the right tumor antigen as a target is the key to designing a safe and effective CAR-T cell. Since CD19 is expressed only in normal and malignant B cells at various stages of differentiation and not on other non-B cells (such as hematopoietic stem cells), it is a potential target for the treatment of B-lineage tumors and a hot spot in CAR-T research. Thus, CD19CAR-T is widely used for malignancy such as acute B lymphocytic leukemia (B-ALL), chronic B lymphocytic leukemia (B-CLL), mantle cell lymphoma (MCL), NHL, and multiple myeloma (MM). A CD19CAR-T has been used in a clinical trial treatment of B cell lymphoma.

PD-1 (Programmed Death 1, reprogrammed cell death receptor 1) is a member of the regulatory T cell CD28 family and belongs to the immunoglobulin receptor superfamily. PD-1 and its ligand PD-L1/PD-L2 play important roles in the co-suppression and failure of T cells. Their interaction inhibits the proliferation of co-stimulatory T cells and the secretion of cytokines. The expression of the anti-apoptotic molecule BCL-xl impairs the function of tumor-specific T cells, leading to the inability of some tumor patients to completely eliminate the tumor. Anti-PD-1 antibody competes with the ligand PD-L1/PD-L2 for binding the PD-1 molecule of the tumor-specific T cell surface, thereby inhibiting complexation of PD-1 and PD-L1/PD-L2. This in turn overcomes the immune microenvironment inhibition caused by PD-1 complexation by PD-L1/PD-L2.

Currently commercialized anti-PD-1 antibodies are nivolumab and pidilizumab. These two monoclonal antibodies have been shown to have good clinical efficacy in solid tumors such as melanoma, colon cancer, prostate cancer, non-small cell lung cancer, and renal cell carcinoma. Recent clinical studies have confirmed that PD-1 antibody can be used in lymphoma therapy. However, anti-PD-1 antibodies still have some unavoidable problems in clinical applications. On the one hand, because anti-PD-1 monoclonal antibody is administered intravenously, most patients receiving PD-1 antibody blockade will have different degrees of drug administration side effects. Also, in vitro production of anti-PD-1 monoclonal antibody involves a complicated production preparation and purification process, which is costly and leads to expensive treatment.

In summary, CAR-T cells have the ability to kill tumor cells and can effectively enter the tumor tissue, but their activity is easily inhibited in the tumor microenvironment; and PD-1 antibody can reactivate the antitumor activity of T cells. However, conventional macromolecular antibodies or large fragments thereof have insufficient power to penetrate into solid tumors, and systemic drugs have large toxic side effects, and the cost of drugs is high.

Therefore, a solution to this problem is disclosed herein, in which an anti-PD-1 antibody can be efficiently expressed by maintaining the killing toxicity of CAR-bearing immune cells (e.g. a CAR-T cell), and the PD-1 antibody is expressed at a high level in or near the tumor by the CAR-bearing cell. This activity is expected to increase the tumor-killing efficacy of the CAR-bearing cells, while also reducing treatment costs.

Presently disclosed is a system having some features similar to CAR-T, but of a more generalized nature. Also, by including an extracellular (perhaps synthetic and of a not naturally-occurring amino acid sequence) peptide molecule having bispecific binding activity of binding both an effector cell bearing a CAR and a target cell bearing a cell surface antigen as an “Immune Cell Activator Polypeptide” (ICAP), the activity level of the CAR-bearing effector cells can be modulated by control of the amount of the ICAP available to bind the CAR. Such a system can be applied to solve the problem of high tonic activity exhibited by CAR-T cells of the prior art.

Some of the terms related to the present disclosure are explained below.

In the present disclosure, the term “expression cassette” refers to the entire element required for expression of a gene, including a promoter, a coding sequence, and a polyA tailing signal sequence.

The term “coding sequence” is defined herein as a portion of a nucleic acid sequence that encodes the amino acid sequence of a polypeptide product (eg, a CAR, a Single Chain Antibody or a domain thereof). The boundaries of the coding sequence are typically determined by a ribosome binding site (for prokaryotic cells) immediately upstream of the open reading frame of the 5′ end of the encoded mRNA and a transcription termination sequence immediately downstream of the open reading frame of the 3′ end of the encoded mRNA. A coding sequence can include, but is not limited to, DNA, cDNA, and recombinant nucleic acid sequences.

The term “Fc”, (fragment crystallizable) is a portion of a mammalian antibody, and refers to a peptide located at the end of the handle of the “Y” structure of the antibody molecule, comprising the CH2 and CH3 domains of the heavy chain constant region of the antibody, and is the site of many molecular and cellular interactions that provide some of the biological effects of a mammalian antibody.

The term “costimulatory molecule” refers to a molecule that is present on the surface of an antigen presenting cell and that binds to a costimulatory molecule receptor on a Th cell to produce a costimulatory signal. The proliferation of lymphocytes requires not only the binding of antigens, but also the signals of costimulatory molecules. The costimulatory signal is transmitted to the T cells mainly by binding to the co-stimulatory molecule CD80 on the surface of the antigen presenting cells, and CD86 binds to a CD28 molecule on the surface of the T cell. B cells receive a costimulatory signal that can pass through a common pathogen component such as LPS, or through a complement component, or through activated antigen-specific Th cell surface protein CD40L.

The term “linker” is a polypeptide fragment that links between different proteins or polypeptides for the purpose of maintaining the spatial relationship of the linked proteins or polypeptides to maintain the function or activity of the protein or polypeptide, for example by relieving steric inhibition of binding of a ligand. Exemplary linkers include linkers containing glycine and/or serine, as well as, for example, a Furin 2A peptide.

The term “specifically binds” refers to the reaction a binding protein and a ligand, for example as between an antibody or antigen-binding fragment and the antigen to which it is directed. In certain embodiments, an antibody that specifically binds to an antigen (or an antibody that is specific for an antigen) means that the antibody-antigen affinity is characterized by a binding constant, Kd, of less than about 10-5 M, such as less than about 10⁻⁶ M, 10⁻⁷ M, 10⁻⁸M, 10⁻⁹ M or 10⁻¹⁰ M or less. “Specifically recognizes,” or “specific recognition” has a similar meaning.

The term “pharmaceutically acceptable excipient” refers to carriers and/or excipients that are compatible pharmacologically and/or physiologically to the subject and active ingredient, which are well known in the art (see, for example, Remington's Pharmaceutical Sciences, editor Gennaro A R, 19th ed. Pennsylvania: Mack Publishing Company, 1995, hereby incorporated by reference in its entirety and for all purposes), and includes, but is not limited to, pH adjusters, surfactants, adjuvants, ionic strength enhancers. For example, pH adjusting agents include, but are not limited to, phosphate buffers; surfactants include, but are not limited to, cationic, anionic or nonionic surfactants such as Tween-80; ionic strength enhancers include, but are not limited to, sodium chloride.

The term “effective amount” refers to a dose that can achieve a treatment, prevention, alleviation, and/or alleviation of a disease or condition described herein in a subject.

The term “disease and/or condition” refers to a physical state of the subject that is associated with the disease and/or condition described herein.

The term “subject” or “patient” may refer to a patient or other animal that receives the pharmaceutical composition of the invention to treat, prevent, ameliorate and/or alleviate the disease or condition of the invention, particularly a mammal, such as a human, a dog, monkeys, cattle, horses, etc.

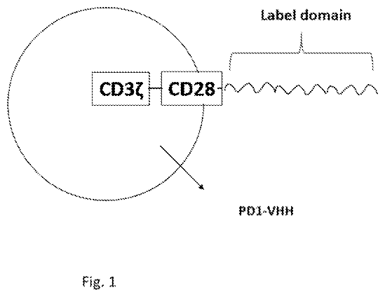

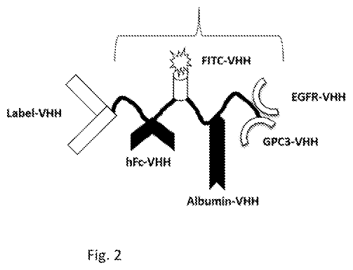

As used herein, a “chimeric antigen receptor” (CAR) is an artificially engineered protein that binds a specific molecule, for example a tumor cell surface antigen, and that stimulates a proliferative program in an immune cell-type effector cell. A CAR typically comprises, in order from amino- to carboxy-terminus, an optional signal peptide (which might be removed during the process of localization of the CAR in the cell membrane of the host cell); a polypeptide that specifically binds to another protein (“label domain”), such as an antigen binding region of a single chain antibody; an optional (but typically present) hinge region; a transmembrane region; and an intracellular signaling region (see, e.g. FIG. 1.) The label domain polypeptide may be one derived from a natural polypeptide or may be a synthetic polypeptide.

In the present application, a “VHH domain” can refer to a variable domain of a single heavy-chain antibody (“VHH antibody”), such as a camelid antibody. A “Single Chain Antibody” (SCA) is a single chain polypeptide and typically includes a number of relatively conserved domains that associate together as the polypeptide folds to form a Framework Region (FR region), and variable regions that associate together to form a variable, antigen-binding domain. A VHH antibody is accordingly a kind of a SCA. In accordance with this terminology, a variable domain present in a naturally occurring single heavy-chain antibody, will also be referred to herein as a “VHH domain”, in order to distinguish such from the heavy chain variable domains that are present in conventional 4-chain antibodies (which will be referred to herein as “VH domains”) and from the light chain variable domains that are present in conventional 4-chain antibodies (which will be referred to herein as “VL domains”).

Isolated single variable domain polypeptides preferably are ones having the full antigen-binding capacity of their cognate SCAs and are stable in an aqueous solution.

Stable, antigen-binding single chain polypeptides comprising one or more domains (of either FR or variable region origin) derived from, or similar to, domains of mammalian antibodies, such as a VH domain, are also encompassed by “Single Chain Antibodies” herein.

A “nanobody” can include a SCA or VHH antibody, or one or more domains thereof, but this word is used more typically to describe an engineered polypeptide comprising one or more VHH domains, and optionally further comprising one or more FR domains, and can additionally or alternatively further comprise additional stable domains that have some further biological activity, such as binding to a flurorophore or binding and activating an extracellular receptor.

Disclosed herein is a novel cell therapy product, an engineered immune effector cell, which can be one such as a so-called “Baize Super Cell,” comprising a chimeric receptor, that can be induced to express a secreted protein in a controllable manner and in situ.

In some embodiments, the engineered immune effector cell constitutively expresses high levels of an effector polypeptide, such as a single chain anti-PD-1 antibody (VHH-PD-1). In some such embodiments, T-cell proliferation activated by binding of a “label domain” of a of an immune effector cell that is a T-cell by a cell surface-associated antigen of a cell provides a very large number of T cells that constitutively secrete an effector polypeptide. In instances where the label domain is bound by an antigen on the surface of a tumor cell, an immune modulatory effector polypeptide can be one that is constitutively expressed and, by virtue of being secreted in the vicinity of the tumor cell, alleviates or avoids immunotolerance induced by e.g. PD-1:PD-L1/L2 complex formation.

Additionally or alternatively, an engineered effector cell as disclosed herein can be engineered to comprise a nucleic acid vector that comprises a coding sequence construct encoding one or more “effector polypeptides” that is expressed under control of a promoter that is operable in an immune cell and that further comprises transcription termination sequences operable in an immune cell. The promoter can be a constitutive promoter, such as a EF1a promoter or a CMV promoter.

The nucleic acid vector can be a retroviral vector or a lentiviral vector. The nucleic acid vector can be a DNA or RNA vector. The vector can comprise a PiggyBac (PB) transposon or a SleepingBeauty (SB) transposon or portion thereof. The vector can comprise transposon-specific Inverted Terminal Repeat sequences, which are typically located at both ends of a transposon-based vector.

An engineered effector cell as disclosed herein can be one in which either or both of an expression cassette encoding an ICAP and an expression cassette encoding one or more effector polypeptides are integrated into the nuclear genome of the effector cell.

A protein to be secreted by the effector cell can be one that is immunostimulatory, such as a polypeptide that specifically binds 4-1BB or OX40, or immuno-inhibitory (for example so as to treat an allergy response or an arthritic condition), such as a polypeptide that specifically binds TNF-α or IL-6.

A preferred protein to be secreted by the effector cell is an antibody or a fragment thereof, or a polypeptide that is a single chain-single domain polypeptide, for example a VHH nanobody or scFv protein. One class of proteins that can be secreted is an immune checkpoint receptor antagonist or agonist antibody with or without a Fc domain. However, other proteins might be expressed and secreted by an engineered effector cell, such as cytokines or another immunomodulatory protein. For example, an antibody, an antigen-binding portion of an antibody or a single chain antibody, such as a VHH nanobody, against PDL1, CTLA-4, CD-40, LAG-3, TIM-3, BTLA, CD160, 2B4, CD40, 4-1BB, GITR, OX-40, CD27, HVEM or LIGHT can be expressed and secreted from an effector cell. Examples of secreted cytokines from an effector cell may include TGF-β, VEGF, TNF-α, CCR5, CCR7, IL-2, IL-7, IL-15 and IL-17. An engineered effector cell of the present invention can express and secrete two or more different types of effector polypeptides, including different antibodies, cytokines, or combinations thereof. As an example, an engineered effector cell can secrete an anti-PDL1 antibody and an anti-CTLA-4 antibody, or an anti-PDL1 antibody and VEGF antibody.

An example of a secreted effector protein is an anti-PD-1 VHH antibody (1182) having an amino acid sequence

(SEQ ID NO:3)
QVQLVESGGGLVQAGGSLRLSCAASGDTSFISAAGWYRQAPGKERELVA
AITNTGITYYPDSVKGRFTISRDNAKNTVYLQMNNLKPEDTAVYYCNAG
APPPGGLGYDESDYWGQGTQVTVSS.

The host immune cells that are engineered can be various T-cells, CIK (cytokine induced killer cells), DC-CIK (dendritic cell/CIK), NK (natural killer cells), NKT (natural killer T cells), stem cell, TIL (tumor infiltrating lymphocytes), macrophage and other immune cells. The host immune cells are typically autologous cell of a subject being treated for a disease.

In some embodiments, the engineered immune cells are transformed with a vector comprising a coding sequence construct having at least 3 structural components: a polynucleotide encoding a first domain that includes intracellular signaling domains that activate a transcriptional program in an “activated” T cell, for example, a CD3ε (CD3e) or a CD3ζ (CD3z) domain of a T-cell surface glycoprotein; a second polynucleotide encodes a domain that contains a transmembrane domain (and optionally spacer peptides), for example one from a CD28 protein; and third polypeptide that encodes a domain that is a “label” polypeptide, specific binding of which by another polypeptide activates a transcriptional program in a host immune cell, such as a T cell via the intracellular signaling domain.

The intracellular signaling domain can include domains that participate in immune co-stimulatory signaling (for example a B7 binding domain), and additionally or alternatively an ITAM domain of a CD3e. Preferably an ITAM domain includes an amino acid sequence YMNM (SEQ ID NO:4).

In some embodiments, both of a transmembrane domain and an intracellular signaling domain are those of a CD28 protein.

In some embodiments, the signal transduction domain comprises an immune co-stimulatory domain joined to a CD3e domain, such as CD28/CD3e, 4-1BB/CD3e, ICOS/CD3e, CD27/CD3e, OX40/CD3e or CD40L/CD3e.

The label domain polypeptide is preferably one that is not expressed, or is minimally expressed, in adult human tissues. For example, the label polypeptide might be derived from a protein that only expressed, or expressed predominantly, in embryonic human cells (i.e., a “fetoprotein”) or a label polypeptide can be a completely synthetic amino acid sequence.

Examples of fetoproteins from which a label polypeptide might be derived include fetoproteins expressed during embryogenesis such as Oct-4, Sox-2, and Klf-2. In some embodiments, less than the complete full-length protein is used; typically between 20-100 aa long polypeptides are used. Below are the amino acid sequences for Oct-4, Sox-2 and Klf-2:

Oct4:
(SEQ ID NO:5)
MAGHLASDFAFSPPPGGGGDGPGGPEPGWVDPRTWLSFQGPPGGPGIGP
GVGPGSEVWGI
Sox-2:
(SEQ ID NO:6)
MYNMMETELKPPGPQQTSGGGGGNSTAAAAGGNQKNSPDRVKRPMNAFM
VWSR
Klf-2:
(SEQ ID NO:7)
MALSEPILPSFSTFASPCRERGLQERWPRAEPESGGTDDDLNSVLDFIL
SMGLD

A label domain portion of an ICAP can be a polypeptide having an amino acid sequence MAGHLASDFAFSPPPGGGGDGPGGPEPGWVDPRTWLSF (SEQ ID NO:8).

The ICAP label domain may contain a structurally inert domain from human mesothelin ECD. For polypeptides encoding the mesothelin domain, it may contain peptide sequences from domain I, II or III as follows:

(domain II - SEQ ID NO:9)
EVEKTACPSGKKAREIDESLIFYKKWELEACVDAALLATQMDRVNAIPF
TYEQLDVLKHKLDEL
(domain II - SEQ ID NO:10)
SLETLKALLEVNKGHEMSPQVATLIDRFVKGRGQLDKDTLDTLTAFYPG
YLCSLSPEELSSVPPSSIWAVRPQDLDTCDPRQLDVLYPKARLAFQN
(domain III - SEQ ID NO:11)
CSLSPEELSSVPPSSIWAVRPQDLDTCDPRQLDVLYPKARLAFQNMNGS
EYFVKIQSFLGGAPTEDLKALSQQNVSMDLATFMKLRTDAVLPLTVAEV
QKL

A label polypeptide can be derived from a structural membrane protein that has no intracellular signal transduction function or interaction with other bioactive molecules, to provide a “structurally inert” domain of the structural membrane protein provided it is one that is normally bound by another protein or carbohydrate and so the epitope constituting the label domain is not exposed to antibodies in vivo. The label polypeptide preferably has little or no immunogenicity. Immunogenicity of the label polypeptide can be determined by 1) in silico computational algorithms on the number of T-cell epitopes 2) in vitro assays to determine T cell activation potential, and 3) in vivo experiments using animal models.

Any label domain as described above can be combined with any transmembrane domain described above and any intracellular signaling domain as described above to form an ICAP polypeptide. Short polypeptide linkers can be used to join domains of an ICAP.

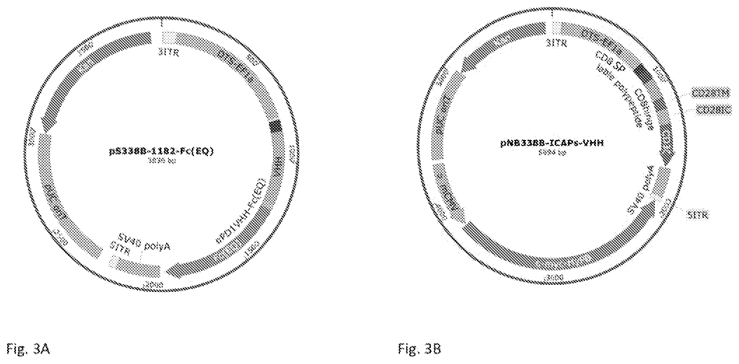

For example, any of the label domains described above can be encoded as the “label domain” portion of the plasmid pNB338B-ICAPs-VHH shown in FIG. 3B.

The construct is expressed in the effector cell to produce a “immune cell activating polypeptide” (ICAP), that localizes to the outer cell membrane such that the label domain is extracellular.

An effector cell as disclosed herein can be used with a bispecific polypeptide—that is, polypeptide having two functional domains joined by a joining polypeptide, or by chemical conjugation, each domain having activity of specifically binding a different ligand. Herein, in some embodiments, the bispecific polypeptide is also called a “VHH-TCP”, as a preferred form for the bispecific polypeptide comprising two or more single chain nanobodies (single chain, single domain antibodies).

One domain of the bispecific polypeptide comprises an amino acid sequence that specifically binds to the label domain of an ICAP on the surface of an effector cell (L-bd), and one domain of the bispecific polypeptide specifically binds to a protein on the surface of a “target”, which is preferably a cellular target, such as a tumor cell, but might be any cell or surface bound with the target protein (CSP-bd). Such a surface presented target polypeptide is called herein a “cell surface protein” or an epitope thereof.

Such cell surface proteins can be antigens associated with tumors, auto-immunity disease, or cellular or organismic senescence; e.g. CD19, mesothelin, BCMA, EGFR, vimentin, Dcr2 or DPP4. In some embodiments, the target cells are cells that abnormally express one or more of these proteins, either as to amount or as to a mutated protein; e.g. a tumor of a B cell, mesothelial cell, breast cell, or fibroblast cell.

The bispecific polypeptide (VHH-TCP), as used herein, can include additional domains, to provide additional binding, or biochemical or physiological activities, for example to recognize multiple epitopes either from the same target protein, or epitopes from multiple target proteins (a “polyspecific polypeptide”, which includes, for example, a tri specific, tetraspecific, pentaspecific or hexaspecific polypeptide). The bispecific polypeptide can also include one or more binding motifs to recognize a human IgG Fc domain as a label domain of an ICAP to effect an effector cell activity-switcher through ADCC, CDC and ADCP mechanisms.

Additionally or alternatively, the bispecific (polyspecific) polypeptide (VHH-TCP) can also include one or more domains derived from serum albumin with various molecular weights to control the half-life of the bispecific polypeptide in vivo.

A domain for binding a fluorophore can be included in a bispecific polypeptide to allow tracking in vivo (e.g. by examination of fluorophore-stained tissue samples) of the bispecific polypeptide and of cells to which the bispecific polypeptide is specifically bound.

Preferably, the domains of the bispecific polypeptide can be joined to one another N-terminal to C-terminal by one or more linker peptides. The length of linkers can be adjusted to tune the molecular weight of the bispecific polypeptide or steric interactions among its domains (e.g. to lessen them).

Linker portions of a bispecific polypeptide can also include amino acid sequences that are susceptible to cleavage by peptidases in the blood, thereby limiting the half-life of the bispecific polypeptide in the blood or extracellular matrix. For example, the amino acid sequences RVLAEA (SEQ ID NO:12), EDVVCCSMSY (SEQ ID NO:13) and GGIEGRGS (SEQ ID NO:14) are cleavable by matrix metalloproteinase-1, and the amino acid sequence VSQTSKLTRAETVFPDV (SEQ ID NO:15) is cleavable by Factor IXa/Factor VIIa.

In some embodiments, one or more, e.g. all, of the active domains are comprised of VHH nanobody polypeptides.

A L-bd can be a single antibody domain derived from the VHH domain of a camelid IgG. The CDR3 region of such a VHH domain can contain 15-20 amino acids that serve as the paratope binding to epitope(s) on the label domain.

The bispecific polypeptide can comprise a L-bd that is a VHH domain that specifically binds a label polypeptide and a CSP-bd that is a VHH domain that specifically binds to CD19 or CD20. Such a bispecific polypeptide would be useful in treating a B-cell lymphoma, such as a non-Hodgkin's lymphoma. In some embodiments, a bispecific polypeptide can comprise a L-bd that is a VHH domain that specifically binds a label polypeptide and a CSP-bd that is a VHH domain that specifically binds to EGFR. An amino acid sequence from the CDR3 region of the VHH antibody can bind to EGFR of on the surface of non-small cell lung cancer cells. Such bispecific polypeptides would be useful in treating a non-small cell lung cancer.

A bispecific polypeptide can comprise a L-bd that is a VHH domain that specifically binds a label polypeptide and a CSP-bd that is a VHH domain that specifically binds to CPC3. In some embodiments, a bispecific polypeptide can comprise a L-bd that is a VHH domain that specifically binds a label polypeptide and a CSP-bd that is a VHH domain that specifically binds to BCMA. In some embodiments, a bispecific polypeptide can comprise a L-bd that is a VHH domain that specifically binds a label polypeptide and a CSP-bd that is a VHH domain that specifically binds to HER2. Such a bispecific polypeptide would be useful in treating a HER2⁺ breast cancer tumor.

An exemplary bispecific polypeptide that comprises two VHH domains joined by a linker (VHH that binds to a label domain containing a structurally inert peptide derived from the human mesothelin's ECD+linker+anti-EGFR VHH) has an amino acid sequence

(SEQ ID NO: 16)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSGGGGSGGGGSQVQLVE
SGGGLVQPGGSLNLSCAASGFDFSSVTMSWHRQSPGKERETVAVISNIG
NRNVGSSVRGRFTISRDNKKQTVHLQMDNLKPEDTGIYRCKAWGLDLWG
PGTQVTVSS.

Preferably binding of the bispecific polypeptide to epitopes on other cells than on the target cells does not have significant impact on the pharmacokinetics or pharmaco-distribution of the bispecific polypeptide in vivo, and preferably such binding as does occur does not cause any significant observable physiological effects other than activating the effector cell expressing the associated label domain to be bound by the bispecific polypeptide.

By virtue of the embodiments illustrated and described herein, Applicant has devised a method and variations thereof for treating tumors using the engineered effector cells and bispecific polypeptides disclosed herein.

In one such method the engineered immune cells, which can be T-cells, or other cells types as described herein as effector cells, are injected directly into a solid tumor. Alternatively, the engineered immune cells can be administered intravenously (IV, e.g. when a leukemia or lymphoma is treated). Different administration methods may be performed depending on the disease indication. In most cases, IV administration is performed to treat disease. Intraperitoneal administration can be performed to treat malignant pleural mesothelioma (MPM).

For treatment of a solid tumor, direct injection into the tumor is expected to result in better distribution of cells within the tumor microenvironment (higher amounts of the engineered immune cells in proximity to the target tumor cells).

In a typical treatment method, the amount of a VHH-TCP to be administered can range from 10 ng/ml to 100 ng/ml together with the engineered immune cells at a concentration of, e.g. 5×10⁴, 1×10⁵, 5×10⁵, or 1×10⁶ engineered cells/ml.

In one example embodiment of a treatment method, which does not utilize a VHH-TCP activating molecule, the engineered immune cells are T cells expressing an ICAP having a VHH label domain that specifically binds to CD19 on a B cell and having the transmembrane domain and intracellular signaling domains of the common T-Cell Receptor (i.e. CD28 and CD3e). The engineered T-cells also comprise a vector for expression of an anti-PD-1-Fc effector polypeptide under control of a constitutive promoter. After administration of the cells to a subject, the label-VHH domain of the ICAP specifically binds to CD19 on B cells, and the binding transduces signals to the engineered immune cells which are then activated upon CD3 and CD28 intracellular signaling and proliferate in the vicinity of the B cell target. The proliferating cells secrete a large amount of the anti-PD1 effector protein in the vicinity of the bound B cell.

The disclosed system, in its various embodiments, provides one or more of the following advantages. Not every embodiment will exhibit every one of the advantages set out below.

In embodiments of the ICAP label domain, a polypeptide derived from fetoprotein or structural membrane proteins provides a wide range of possible L-bds for a VHH-TCP binding, and may improve cell therapy safety due to no or less immunogenicity of the domain.

The diversity of domains that can be included in bispecific polypeptide (VHH-TCP) provides the ability to alter many properties such as VHH-TCP affinity to the effector cell, and the range of cells that can be targeted by the CSP-bd is broad. Also other functional domains can be added, and epitope binding valency can be adjusted to efficacy and safety of use of the system to treat a disease.

A bispecific polypeptide (VHH-TCP) that specifically binds to a label domain of an immune cell activator polypeptide that includes a signaling domain that activates proliferation of the immune cell host and specifically binds to a cell surface protein of B cells such as the CD19 ligand, can induce proliferation of the effector cells in vivo increase the population of the immune effector cells, thus increasing the amount of the effector polypeptide, in the B cell binding vicinity. This allows for time savings and avoidance of the costs of in vitro production of the effector polypeptide.

A bispecific polypeptide (VHH-TCP) can be engineered in various formats to optimize the effector cell activity through the length and flexibility of linkers among VHHs in the bispecific polypeptide (VHH-TCP), the position of each binding motif and the overall size of VHH-TCM.

Effector cell activity in vivo can be controlled by dosing with different amounts of a bispecific polypeptide (VHH-TCP), and/or controlling the half-life of a VHH-TCP. This is a novel and comprehensive approach to minimize the toxicity of a CAR-based therapy.

Furthermore, using an appropriate label protein that specifically binds Fc epitopes, effector cells can be activated by ADCC effects.

Importantly, the characteristics of a nanobody such as small size, high stability and easy engineering offers unique advantages for optimizing a treatment system in vivo.

Discontinuation of VHH-TCP administration to a subject can prevent adverse effects associated with the persistent effector cell activity while also providing the opportunity for subsequent VHH-TCP administration in the event of disease relapse.

In situ secretion of antibody, preferably nanobody, by activated effector cells can either inhibit or stimulate immune checkpoint receptors to improve targeting of solid tumors through TME (tumor microenvironment) penetrance, proliferation and persistence. Nanobody bispecific polypeptides have advantages over conventional antibodies in penetrating the TME due to their small size and great stability.

The effector cell—bispecific polypeptide (VHH-TCP) system disclosed herein can improve upon many of the pitfalls that accompany current CAR-T therapy: for instance by targeting multiple tumor antigens with a single, standardized immune receptor, and diverse VHH-TCP structures can be used to control and optimize immune cell activity. Treatments utilizing the disclosed system are expected to exhibit less toxicity or side-effects. Further, the components of the system are easily and cost effectively manufactured. The diversity of ligands and binding domains that can be incorporated into the ICAP and bispecific polypeptide (VHH-TCP) allows a modular system to be used to treat a broad variety of diseases or to conduct research, for example incorporating a FITC-binding domain into a VHH-TCP allows the fate of activated effector cells to be followed in vivo.

### EXAMPLES

### Example 1—A Representative Working Embodiment

1. Generation of Modified Effector T Cells by Electroporation

An ICAP comprises of a label polypeptide (27 aa of mesothelin, or fetoproteins), a CD28 transmembrane domain, a CD28 intracellular co-stimulatory signaling domain (CD28IC) and a CD3ζ. The 1182-Fc(EQ) comprises of VHH-1182 and IgG4 Fc domain.

A 1182-Fc(EQ) structural gene is cloned into the piggyBac transposon vector pS338B to obtain a plasmid pS338B-1182-Fc(EQ) (FIG. 3A). An ICAP-VHH gene is PCR amplified and cloned into a piggyBac transposon vector pNB338B, to obtain the plasmid pNB338B-ICAP-VHH (FIG. 3B). The ICAP-VHH gene is replaced by empty multiple clone (MCS) gene to generate a MOCK construct plasmid.

Human Peripheral blood mononuclear cells (PBMCs) of healthy donors are purchased from AllCells (Shanghai, China). PBMCs are cultured in AIM-V medium supplemented with 2% fetal bovine serum (FBS; Gibco, USA) at 37° C. in a 5% CO₂ humidified incubator for 0.5-1 hr, and then harvested and washed twice using Dulbecco's phosphate-buffered saline (PBS).

PBMCs are counted and electroporated with 6 μg pNB338B-ICAP-VHH plasmid or an equal quantity of MOCK plasmid in electroporators (Lonza, Switzerland) using a Amaxa® Human T Cell Nucleofector® Kit according to the manufacturer's instructions. Thereafter, T cells transfected with ICAP-VHH/1182-Fc (EQ) plasmids or MOCK/1182-Fc(EQ) plasmids are specifically stimulated in 6-well plates, which are coated with anti-CD3 antibody/anti-CD28 antibody (5 μg/mL), for 4-5 days. Transformed T cells are then cultured in AIM-V medium containing 2% FBS and 100 U/mL recombinant human interleukin-2 (IL-2) for 10 days to generate a sufficient quantity of effector T cells.

2. Transduction Efficiency Assay

Transduction efficiency of label polypeptide into T cells is determined by flow cytometry using biotin-conjugated anti-IgG4(Fc) antibody and a PE-conjugated streptavidin secondary antibody.

3. Binding Efficiency Assay

The binding of bispecific polypeptide to common T cells is measured by flow cytometry using anti-CD19-PE antibodies. The ratio of cells positive for CD19 and label (e.g. meso) is compared to determine the binding efficiency.

4. Proliferation Ability Assay (in Culture with Bispecific VHH and Tumor Cells)

1×10⁷ transformed T cells are prestained with carboxyfluorescein succininmidyl ester for 10 minutes and recovered in medium for another 10 minutes. 5×10⁵ cells are counted and cocultured with tumor cell lines that express different antigens, including BCMA, EGFR, Mesothelin, MUC1 and GPC3, as well as bispecific VHH for 7 days, replacing the culture medium every 3-4 days with fresh medium (AIM-V+2% FBS). The effector cells are then assayed for proliferation by flow cytometry.

5. Quantification of 1182-Fc-VHH Secretion

5×10⁵ cells are seeded at in 6-well plates with 1 ml medium, and tumor cells and bispecific VHH are added and cocultured for 48 hours. Then the effector T cell suspension is centrifuged at 3000 rpm for 3 min; the supernatant is retained and 1182-Fc protein is quantitatively detected by ELISA.

6. Cytotoxicity Assay (for Adhesion Cell Lines)

The cytotoxicity of effector T cells transduced with label construct or vector control is determined by using an impedance-based xCELLigence RTCA TP Instrument.

Target tumor cells are seeded in a resistor-bottomed 96-well plate at 10,000 cells per well within the RTCA TP instrument overnight (more than 16 hours). The bispecific VHH antibody is added to the cultured target tumor cells and the cells are further cultured for 30 minutes. Then transformed T cells harboring the plasmids pS338B-1182-Fc(EQ) and pNB338B-ICAP-VHH (effector cells) are incubated with target tumor cells at different effector cell:target cell ratios for about 100 hours (the end point depends on the killing efficiency of transformed T cells). During the experiment, the cell index values are closely correlated with tumor cell adherence, such that lower cell attachment indicates higher cytotoxicity, and are collected every 5 minutes by the RTCA system and an EnVision® Multilabel Plate Reader (PerkinElmer). The real-time killing curves are automatically generated by the system software. Specific lysis (%) of each transformed T cell are also calculated using the data of the end point [specific lysis=(cell index of tumor cells alone−cell index of transformed T cells cocultured with tumor cells)/cell index of tumor cells alone].

7. Cytotoxicity Assay (for Suspension Cell Lines)

The cytotoxicity of effector T cells transduced with label construct or vector control is determined according to the manufacturer's protocol (DELFIA® EuTDA Cytotoxicity Reagents AD0116—PerkinElmer). Briefly, target tumor cells are washed with PBS and fluorescence enhancing ligand and are incubated for 5-30 minutes at 37° C. 100 ul of target cells (10,000 cells) are placed into a V-bottom plate containing bispecific polypeptide that specifically binds to both of the target tumor cells and to the effector cells (that is, the transformed T cells), and 100 ul of effector cells are added with varying cell concentration. 20 ul of the supernatant were transferred into 200 μL of Europium Solution following 15 minutes incubation at room temperature. The fluorescence is measured in the time-resolved fluorometer. Specific release (%)=Experimental release (counts)−Spontaneous release (counts)/Maximum release (counts)−Spontaneous release (counts)×100.

8. Specific Targeting Activity In Vivo

NOD-SCID IL2 Ry^{-/-} (NSG) mice are raised under the pathogen-free condition (Shanghai, China). The animal experiments are approved by Institutional Animal Care and Use Committee (IACUC). To establish xenograft tumor models, NSG mice are subcutaneously inoculated with 5×10⁶ EGFR⁺ lung tumor cells and 5×10⁶ MSLN⁺ ovarian tumor cells mixed with an equal volume of Matrigel™. The tumor dimensions are obtained using vernier calipers, and the tumor volumes are calculated based on the formula: V=½(length×width²). When the tumor burdens are approximately 100 mm³, the Fluc-labelled effector T cells and bispecific EGFR-targeting VHH are intravenously injected. The specific targeting of effector T cells to lung is confirmed by bioluminescence imaging (BLI). On day 5, another bispecific MSLN-targeting VHH is intravenously injected to observe the specific targeting of effector T cells to ovarian tumor cells. The proliferation ability of effector T cells in vivo is monitored by bioluminescence imaging using a Xenogen IVIS imaging system (PerkinElmer, USA).

9. Proliferation and Antitumor Activity In Vivo

To establish xenograft tumor models, NSG mice are subcutaneously inoculated with 5×10⁶Fluc-labelled tumor cells mixed with an equal volume of Matrigel™. When the tumor burdens are approximately 100 mm³, mice are randomly separated into three groups (5 mice per group) for intravenous injection (i.v.) of MOCK-T, effector T cells, or PBS vehicle with polypeptides VHH, the time point of which is designated as day 0.

Peripheral blood of all mice is taken from the tail vein to detect the proliferation of effector T cells and copy numbers of ICAP gene. Mice are euthanized after a moribund state is reached, and then the bone marrow, blood and spleen are collected. The percent of CD3⁺ T cells in the above tissues and the memory T cell subsets in spleens are analyzed by flow cytometry. During the whole experimental progress in vivo, mice body weight is measured using an electronic balance. The tumor progression is confirmed by bioluminescence imaging (BLI) using a Xenogen IVIS imaging system (PerkinElmer, USA). All measures are conducted every five days.

10. Hematoxylin-Eosin (H&E) Staining and Immunohistochemistry (IHC)

H&E and immunohistochemistry are performed to evaluate the safety of the cell therapy. Mouse tissues (heart, liver, spleen, lung, kidney and brain) are fixed with formalin and then embedded with paraffin. The tissues are cut into 4 μm thickness consecutively using a R1\42245 microtome (Leica, Germany), and then stained with H&E. To detect the infiltrative ability of the effector T cells within tumor tissues, IHC analysis is performed using anti-CD3 antibody (Abeam, #ab16669) at 1:100 dilution. Images are taken with an AXIOSTAR PLUS microscope (ZEISS, Germany).

11. Tissue Distribution Assay

The tissue distribution of 1182-VHH, transfected T cells and adaptor VHH proteins is determined using Quantitative Real-time PCR (RT-qPCR). Mouse tissues (heart, liver, spleen, lung, kidney and brain) are digested to prepare single cell suspensions. Total DNA is extracted from T cells using Genomic DNA Extraction Kit Ver. 5.0 (TAKARA, China) according to the manufacturer's instruction. Real-time polymerase chain reaction is performed using TaqMan™ Universal Master Mix II (ThermoFisher Scientific, USA). Primers and probes of CAR and actin are synthesized or labeled by Shanghai Generay Biotech Co. Ltd (Shanghai, China). Quantitative real-time PCR reaction is performed in two steps: (1) Pre-incubation: 95° C. for 5 minutes; (2) Amplification: 40 cycles of 95° C. for 20 seconds followed by 60° C. for 1 minute. All reactions are performed in triplicate.

12. Statistical Analysis

All data are presented as mean±SD. T-test is used to evaluate differences between two independent groups. One-way ANOVA is used to compare whether there are any statistically significant differences between three or more independent groups. Two-way ANOVA is used to determine the effect of two nominal predictor variables on a continuous outcome variable. All statistical analysis is performed using Graphpad Prism 7 version software (La Jolla, Calif.). All data with error bars are presented as mean±SD. Statistics significant difference is considered as follows: P≥0.05 (ns), P<0.05 (*), P<0.01 (**), P<0.001 (***), P<0.0001 (****).

### Example 2. Identification and Characterization of One VHH Sequences with High Affinity Towards MSLN, BCMA or EGFR

The identification and characterization of one specific VHH nanobody towards MSLN, BCMA or EGFR with high affinity using an alpaca immune library is described.

1. VHH Nanobody Towards MSLN

For the first immunization, 400 μg MSLN-hFc emulsified using Freund's complete adjuvant was subcutaneously administered to each alpaca. Two weeks later, 200 μg MSLN-hFc emulsified using Freund's incomplete adjuvant was subcutaneously administered. After that, 5 additional immunizations were carried out with 200 μg MSLN-hFc emulsified using Freund's incomplete adjuvant every other week. High serum titer against both MSLN-His antigen as well as HEK293T-MSLN stable cell line was confirmed by ELISA and FACS.

Seven days after the last injection 50 mL of blood were collected, lymphocytes were purified from the sample, RNA was extracted and used for immune library construction. Two rounds of solid protein panning were conducted with MSLN-His antigen followed by ELISA screening and FACS verification. One positive clone named M2339(VHH) was obtained.

The antibody was expressed as a hFc fusion protein, named as M2339(VHH) with the procedure that was described in the patent publication WO2020176815A2 (hereby incorporated by reference in its entirety and for all purposes). The binding affinity of M2339(VHH) towards MSLN antigen was tested by surface plasmon resonance (SPR). First, M2339(VHH) was passed through the sensor chip with protein A immobilized in advance, and the antibody was captured by protein A. Then, five different concentrations of MSLN-His protein were used as the mobile phase, and the binding time and dissociation time were 30 min and 60 min, respectively. Biacore evaluation software 2.0 (GE) was used to analyze the on-rate (kₒₙ), off-rate (k_{off}) and equilibrium constant (K_{D}). As shown in Table 1 below, the affinity of M2339(VHH) towards MSLN antigen was high with the K_{D} of 2.64E-10.

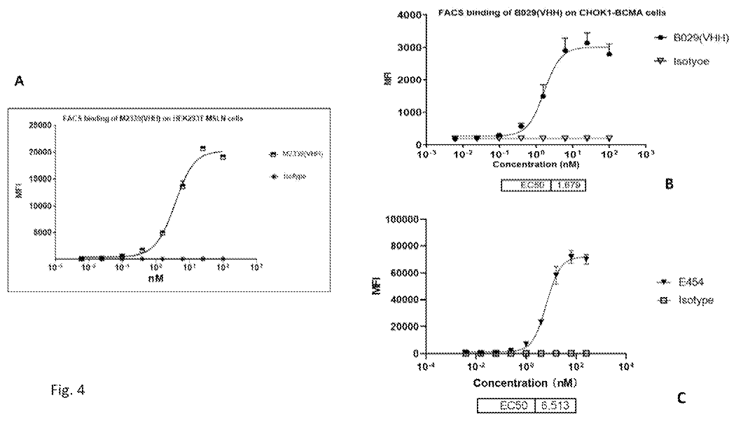

The binding affinity of M2339(VHH) towards HEK293T-MSLN cells was identified with flow cytometry. One 96-well plate was incubated with HEK293T cells and HEK293T-MSLN cells in different wells at 3×10⁵ cells per well, then serially diluted M2339(VHH) was incubated for half an hour, after that the detection secondary antibody anti-human IgG PE (Jackson Immuno Research, Code: 109-117-008) was incubated before detection with CytoFLEX flow cytometer. “Isotype” is an isotype control (negative control). As shown in FIG. 4A, M2339(VHH) showed good and specific binding affinity towards HEK293T-MSLN cell line.

2. VHH Nanobody Towards BCMA

This example describes the identification and characterization of one specific VHH nanobody towards BCMA with high affinity using an alpaca immune library. The procedures for alpaca immunization, blood collection, library construction, solid panning, ELISA or FACS screening for positive clones, antibody purification and followed antibody characterization by SPR and FACS are described above in Example 2. One positive clone named as B029(VHH) was obtained.

As shown in Table 1, the affinity of B029(VHH) towards BCMA-His antigen was high with a K_{D} of 1.25E-10.

As shown in FIG. 4B, B029(VHH) showed good and specific binding affinity towards CHOK1-BCMA cell line.

3. VHH Nanobody Towards EGFR

This example describes the identification and characterization of one specific VHH nanobody towards EGFR with high affinity using an alpaca immune library. The procedures for alpaca immunization, blood collection, library construction, solid panning, ELISA or FACS screening for positive clones, antibody purification and antibody characterization by SPR and FACS are described above in Example 2. One positive clone named as E454(VHH) was obtained.

As shown in Table 1, the affinity of E454(VHH) towards EGFR His antigen was high with the K_{D} of 1.27E-09.

As shown in FIG. 4C, E454(VHH) showed good and specific binding affinity towards HEK293T-EGFR cell line.

TABLE 1
Binding kinetics of M2339(VHH)-MSLN, B029(VHH)-BCMA,
and E454(VHH)-EGFR

| | Kinetics | | |
|---|---|---|---|
| Sample | Ka (1/Ms) | Kd (1/s) | KD (M) |
| M2339(VHH) | 6.90E+04 | 1.82E−05 | 2.64E−10 |
| B029(VHH) | 2.92E+06 | 3.65E−04 | E25E−10 |
| E454(VHH) | 1.95E+04 | 2.48E−05 | E27E−09 |

### Example 3. High Affinity VHH Specific for Region II+III of Mesothelin

This example described the identification and characterization of the label used in this invention that was recognized M2339(VHH) with a similar affinity binding to mesothelin.

Different mesothelin ECD domain with human Fc were expressed in 293T cells and purified by protein A column. Affinity was determined by SPR. Protein A chip was used to capture different antigens, various concentrations of M2339(VHH) were injected at a flow rate of 10 μl/min with an association time of 120 to 180 seconds and a dissociation time from 180 to 1200 seconds. Binding kinetics were determined using Biacore Evaluation software in a 1:1 fit model.

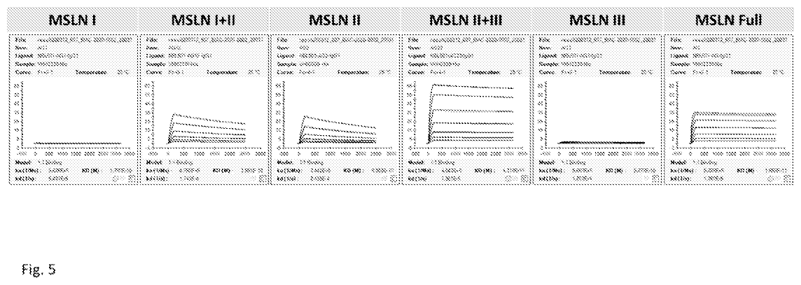

As shown in FIG. 5, M2339(VHH) bound to mesothelin-full, mesothelinI, mesothelinII+III with different affinity. mesothelinII+III domain was well recognized by M2339, with a KD value of 4.32E-11 M, similar affinity to the complete mesothelin polypeptide (Table 2).

TABLE 2
Kinetics of binding of M2339(VHH) to mesothelin domains

| Ligand | Sample | ka (1/Ms) | kd(1/s) | KD (M) | Rmax (RU) |
|---|---|---|---|---|---|
| MSLN I − | M2339 VHH- | 3.63E+05 | 6.49E−05 | 1.79E−10 | 0.3 |
| Fc | 6his | | | | |
| MSLN I + II − Fc | M2339 VHH- | 4.76E+05 | 1.74E−04 | 3.66E−10 | 40.5 |
| | 6his | | | | |
| MSLN II − Fc | M2339 VHH- | 2.44E+05 | 2.44E−04 | 9.99E−10 | 53.3 |
| | 6his | | | | |
| MSLN II + III − Fc | M2339 VHH- | 4.54E+05 | 1.96E−05 | 4.32E−11 | 68.7/30 |
| | 6his | | | | |
| MSLN III − Fc | M2339 VHH- | 5.66E+05 | 1.85E−04 | 3.27E−10 | 1.9 |
| | 6his | | | | |
| MSLN Full − Fc | M2339 VHH- | 8.23E+05 | 1.36E−05 | 1.65E−11 | 35.1/37 |
| | 6his | | | | |

These results demonstrated that M2339 bound mesothelinII+III with a high affinity and could be used as an adaptor VHH, mesothelinII+III can be used in an ICAP in T cells.

### Example 4. Generation and Screening of an Immune Cell Activating Polypeptide Based on Mesothelinii+III (M-ICAP)

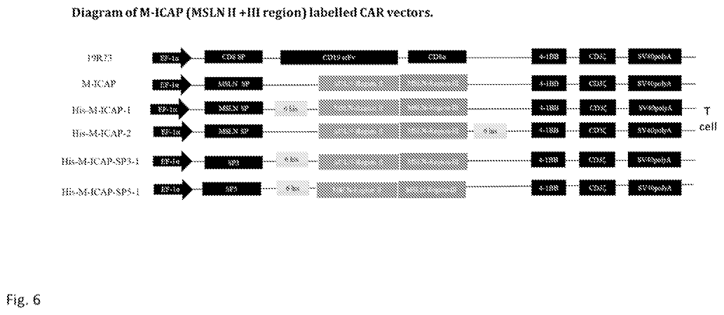

To generate so-called M-ICAP-T, for activating immune cells, e.g. T cells, different vectors were constructed as shown in FIG. 6. All of the vectors encode the same intracellular regions, containing 4-1BB and CD3ζ intracellular regions. The extracellular regions of the encoded polypeptides were different. M-ICAP does not contain any His-tag. M-ICAP-his-1 or 2 contain a 6×His-tag at either the N-end or C-end of M-ICAP, respectively. Besides mesothelin signal peptide (SP-MSLN), two other signal peptides (SP) were selected from human protein database to optimize expression rate. SP3-M-ICAP and SP5-M-ICAP contain different signal peptides SP3 (MKHLWFFLLLVAAPRWVLS—SEQ ID NO:1) or SP5 (MTRLTVLALLAGLLASSRA—SEQ ID NO:2).

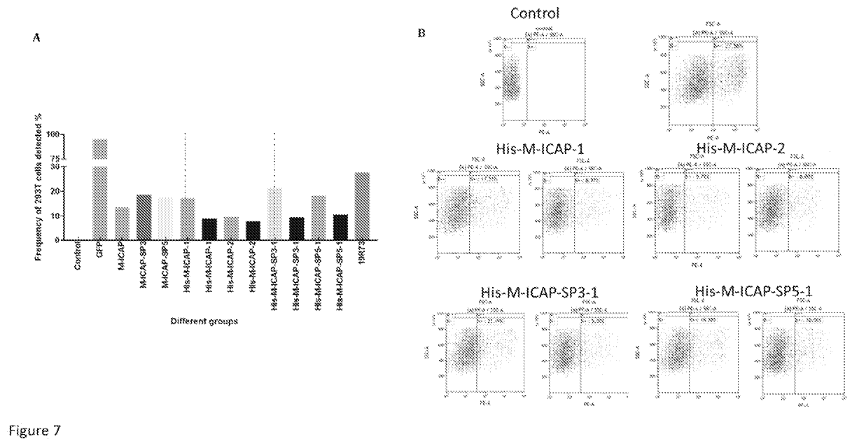

All vectors were transfected into 293T cells with Lipofectamin2000 (ThermoFisher, USA) and after 2-4 days flow cytometry was used to test their expression rate. For flow cytometry detection, a 19R73-CD19CAR and GFP vector was used to prepare a blank cell control, M2339-hFc and biotin conjugated anti-His mAb were used as primary antibody, fluorophore-conjugated anti-human Fc and fluorophore-conjugated streptavidin were used as secondary antibody. As shown in Table 3 and FIG. 7, the location of the His-tag affected label (M-ICAP) expression rate, M-ICAP expression rate was higher when the His-tag at its N terminal is detected by M2339-hFc. Signal peptide showed little effect on M-ICAP expression tested by both M2339-hFc and anti-His.

TABLE 3
Expression levels of various M-ICAP polypeptides

| Plasmid name | Frequency (%) | Detected Antibody |
|---|---|---|
| Control | 0.03 | M2339-IgG1 |
| GFP | 95.44 | / |
| pNB338B-19R73 | 27.56 | anti-CD19-IgG1 |
| pNB338B-M-ICAP | 13.51 | M2339-IgG1 |
| pNB338B-SP3-M-ICAP | 18.66 | M2339-IgG1 |
| pNB338B-SP5-M-ICAP | 17.75 | M2339-IgG1 |
| pNB338B-His-1-M-ICAP | 17.31 | M2339-IgG1 |
| pNB338B-His-1-M-ICAP | 8.97 | Biotin-anti His mAb |
| pNB338B-His-2-M-ICAP | 9.72 | M2339-IgG1 |
| pNB338B-His-2-M-ICAP | 8 | Biotin-anti His mAb |
| pNB338B-SP3-His-M-ICAP | 21.19 | M2339-IgG1 |
| pNB338B-SP3-His-M-ICAP | 9.5 | Biotin-anti His mAb |
| pNB338B-SP5-His-M-ICAP | 18.38 | M2339-IgG1 |
| pNB338B-SP5-His-M-ICAP | 10.56 | Biotin-anti His mAb |

### Example 5. M-ICAP-T Cell Construction

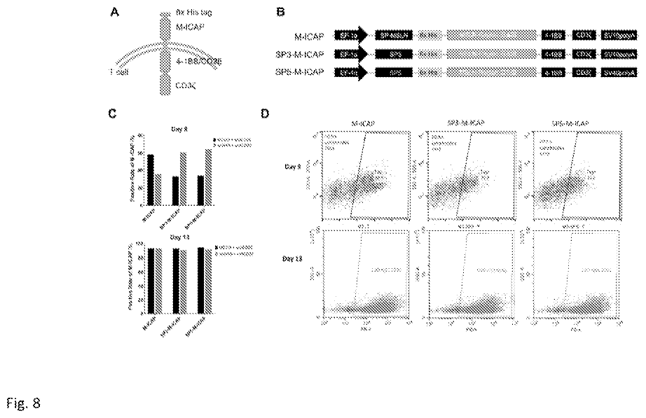

M-ICAP expression vectors containing different signal peptides (SP-MSLN, SP3, SP5) were constructed and fused with the T cell activation/signal transduction domain (CD28/4-1BB, CD3ζ) of traditional CAR vector, as shown in FIG. 8A. The ICAP vector comprised a label polypeptide M-ICAP(from mesothelin II+III domain), a CD28 transmembrane domain, a CD28/4-1BB intracellular co-stimulatory signaling domain (CD28/4-1BBIC) and a CD3ζ domain. The ICAP-VHH gene was amplified by PCR and cloned into a piggyBac transposon vector pNB338B, to obtain the plasmid pNB338B-ICAP (FIG. 8B).

Human Peripheral blood mononuclear cells (PBMCs) of healthy donors were purchased from AllCells (Shanghai, China). PBMCs were cultured in AIM-V medium supplemented with 2% fetal bovine serum (FBS; Gibco, USA) at 37° C. in a 5% CO2 humidified incubator for 0.5-1 hr, and then harvested and washed twice using Dulbecco's phosphate-buffered saline (PBS). PBMCs were counted and electroporated with 6 μg M-ICAP, SP3-M-ICAP and SP5-M-ICAP vectors in electroporators (Lonza, Switzerland) using an Amaxa® Human T Cell Nucleofector® Kit according to the manufacturer's instructions. Thereafter, transfected T cells were stimulated specifically in 6-well plates coated with anti-His/M2339 (VHH-Fc) plus anti-CD28 antibody (5 μg/mL), for 4-5 days, then cultured in AIM-V medium containing 2% FBS and 100 U/mL recombinant human interleukin-2 (IL-2) for 10 days to generate a sufficient quantity of effector T cells. Transduction efficiency of label polypeptide (M-ICAP expression) on T cells was determined by flow cytometry using biotin-conjugated anti-His antibody and a PE-conjugated streptavidin secondary antibody.

As shown in FIGS. 8C and 8D, after expansion the positive rate of M-ICAP-T were all above 30% at Day 8 and 92% at Day 13. All three different ICAPs were activated and amplified by stimulation of M2339 VHH or anti-His antibody. The expression of M-ICAP ECD outside the T cell membrane and M-ICAP-T cell construction were successful.

### Example 6. Generation and Verification of M-ICAP-T Cell

M-ICAP were fused into several different CAR sequences, and obtained M-ICAP-T cells were obtained by electroporation combined with specific activation using donor derived PBMC cells. The ICAP vector comprised a label polypeptide (from mesothelin II+III domain), a CD28 transmembrane domain, a CD28/4-1BB intracellular co-stimulatory signaling domain (CD28/4-1BBIC) and a CD3ζ domain. The 1182-Fc(EQ) comprises VHH-1182 and IgG4 Fc domains.

Generation of cells expressing an ICAP CAR (ICAP-T cells) or of classical CAR-T cells by electroporation was described in Example 5.

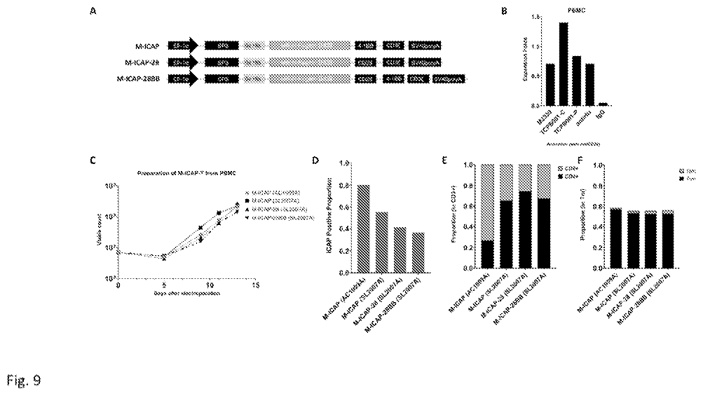

After expansion, a series of tests were carried out to verify the modified T cells, including the positive rate of ICAP expression, the amplification effect, the ratio of CD4/CD8 positive cells in CD3 positive cells and the ratio of effector memory T(Tem)/central memory T (Tcm) cells in memory T cells (Tm). The expression rate of label polypeptide (M-ICAP expression) on the surface of T cells was determined by flow cytometry using biotin-conjugated anti-His antibody and a PE-conjugated streptavidin secondary antibody. As shown in FIG. 9, the amplification of ICAP-T cells during preparation from PBMC of two donors (AC1909A and SL2007A) was up to ten times, and the ICAP expression rate was up to 80% (varying according to different donor sources); the CD4/CD8 positive values varied according to different donors, and central memory T cells accounted for the majority of memory cells. Different CAR-element sequences had some influence on the positive rate and amplification of ICAP-T cells, for example, compared with M-ICAP and M-ICAP-28, M-ICAP-28BB-T cells showed less proliferation and ICAP expression. In terms of specific activation of T cells, we compared the effects of different antibodies/TCPs on the amplification of PBMC transfected with M-ICAP. As shown in the figure, M2339, anti-His antibody and TCP001-C/P could specifically activate the expansion of ICAP-T cells.

### Example 7. Design and Characterization of TCPs Based on M2339 VHH

This example describes the design and characterization of TCPs used in this application. TCPs used here were bispecific antibodies that can simultaneously recognize target B cell or tumor specific antigens (such as CD19, BCMA and EGFR) and M-ICAP polypeptide (from mesothelin) of the M-ICAP-T cells, so they can be used as an adaptor to control the proliferation or cytotoxicity of M-ICAP-T cells. The TCPs designed and applied in the present working Examples are listed in Table 4.

TABLE 4
Domain organization, molecular weight and purity of TCPs used in the Examples

| | N terminal Ab | | C terminal Ab | | | MV | SEC |
|---|---|---|---|---|---|---|---|
| Name | sequence | Linker | sequence | Tag | Name | (kDa) | purity |
| TCP001-C | anti-M-ICAP | 3xGGGGS | anti-BCMA VHH | Myc and | TCP001-C | 30 | 91% |
| | VHH (M2339) | linker | (B029) | HIS | | | |
| TCP002-C | anti-M-ICAP | hIgG4-Fc | anti-BCMA VHH | \ | TCP002-C | 108 | 97% |
| | VHH (M2339) | (S228P) | (B029) | | | | |
| TCP003-C | anti-M-ICAP | hIgG4-CH3 | anti-BCMA VHH | Myc and | TCP003-C | 43 | 98% |
| | VHH (M2339) | | (B029) | HIS | | | |
| TCP-MC | \ | \ | anti-BCMA VHH | Myc and | TCP-MC | 15 | 89% |
| | | | (B029) | HIS | | | |
| TCP-MD | anti-M-ICAP | \ | \ | Myc and | TCP-MD | 16 | 96% |
| | VHH (M2339) | | | HIS | | | |
| TCP001-P | anti-M-ICAP | 3xGGGGS | anti-BCMA scFv | Myc and | TCP001-P | 43 | 99.50% |
| | VHH (M2339) | linker | (b2121) | HIS | | | |
| TCP001-N | anti-M-ICAP | 3xGGGGS | anti-GFP VHH | Myc and | TCP001-N | 30 | 99% |
| | VHH (M2339) | linker | | HIS | | | |
| TCP011-P | anti-M-ICAP | 3xGGGGS | anti-CD19 scFv | Myc and | TCP011-P | 43 | 98% |
| | VHH (M2339) | linker | (FMC063) | HIS | | | |
| TCP021-P | anti-M-ICAP | 3xGGGGS | anti-EGFR VHH | Myc and | TCP021-P | 43 | 96% |
| | VHH (M2339) | linker | (E454(VHH)) | HIS | | | |

1. Design and Purification of TCPs

BCMA-TCPs were designed that can simultaneously target BCMA antigen and M-ICAP polypeptide (label derived from mesothelin) for use in cytotoxicity and in vitro efficacy assays described further below. To investigate the effect of different linker formats on the bioactivity and stability of TCPs, three formats of BCMA-TCPs (TCP001-C, TCP002-C and TCP003-C) with different linkers (3×GGGGS linker, hIgG4-Fc, and hIgG4-CH3 respectively) were designed. TCP001-P and TCP001-N were respectively the positive and negative controls in TCP format. At the same time, MC001C and MC001D, targeting BCMA and M-ICAP respectively, were constructed as two positive controls in mAb format.

One CD19-TCP simultaneously targeting CD19 antigen and M-ICAP polypeptide with the 3×G4S linker, named as TCP011-P, was designed for use in the proliferation assay of M-ICAP-T stimulated by CD19 antigen.

One EGFR-TCP simultaneously targeting EGFR antigen and M-ICAP polypeptide with the 3×G4S linker, named as TCP021-P was designed for use in cytotoxicity assay of M-ICAP-T using EGFR expressing solid tumor cell lines as target.

The N terminal M2339VHH sequence targeting M-ICAP polypeptide was identified from phage display with alpaca immune VHH libraries, described above in Example 2 and Example 3. The B029(VHH) sequence targeting BMCA was identified from phage display with alpaca immune VHH libraries, described above in Example 2. The scFv sequence in TCP001-P targeting BMCA was derived from B2121 of CN201580050638. The VHH sequence in TCP001-N targeting GFP was derived from the GFP-specific VHH described by Kubala et al. (M. H. Kubala et al, Protein Sci. 19:2389-2401 (2010) (hereby incorporated by reference in its entirety for description of such VHH and how it is used).

The scFv sequence in TCP011-P targeting CD19 was derived from FMC063, described in Chinese patent application CN201480027401.4 (hereby incorporated by reference in its entirety and for all purposes). The E454 sequence in TCP021-P targeting EGFR was identified from phage display with alpaca immune VHH libraries, described above in Example 2.

The genes of were synthesized and cloned by Genewiz, Inc. All ORF DNAs were cloned into the pcDNA3.4 vector between the BamHI and EcoRI sites. Antibody expression, purification and purity quality control were conducted as described in the publication WO2020176815A2 (hereby incorporated by reference for description of such methods).

2. Affinity Characterization of TCPs

First the binding affinity of the purified BCMA-TCPs against BMCA antigen was assessed by SPR. BCMA-his antigen was coupled onto a CM5 chip (GE Healthcare Life Sciences), and then a variety of anti-BCMA BsAbs were flowed at the flow rate of 10 uL/min with dissociation time of 900s. Binding kinetics were determined with a 1:1 fit model. The data indicated that linker structure might influence the binding affinity, as TCP001-C with a 3×G45 linker had higher binding affinity than TCP002-C and TCP0031-C with larger linkers (Table 5).

TABLE 5
Kinetics of binding of BCMA-TCPs to BMCA

| | Kinetics | | |
|---|---|---|---|
| Samples | Ka(1/Ms) | Kd (1/s) | KD (M) |
| TCP001-C | 8.82E+05 | 5.18E−04 | 5.87E−10 |
| TCP002-C | 2.69E+04 | 4.82E−04 | 1.79E−08 |
| TCP003-C | 4.23E+04 | 2.98E−04 | 7.04E−09 |
| TCP-MC | 3.96E+06 | 5.46E−04 | 1.38E−10 |
| TCP001-P | 8.25E+05 | 1.25E−05 | 1.51E−11 |

The binding bioactivity for mesothelin and BCMA over-expressing cells was evaluated by flow cytometry. Stable cell lines were harvested using 0.25% Trypsin.

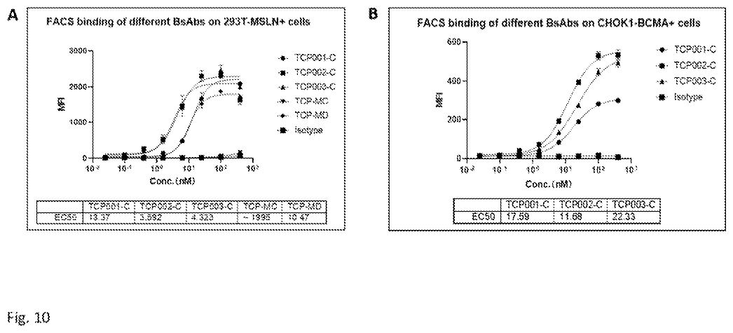

About 5E5 cells were collected per sample, and the cells were resuspended in 100 μL/well tested antibodies with His-tag. Then the cells were incubated with anti-His-tag antibody (Genscript, China) and Streptavidin-PE (Biolegend, China). The incubation step of each step was performed at 4° C. in the dark for 1 hr, and then the cells were washed 2× with 200 μL PBS buffer. The washed cells were resuspended in 200 μL PBS buffer and the sample was analyzed by FACS. As shown in FIG. 10, TCP002-C had the strongest binding to both cell lines, while the binding strength of TCP003-C was a little higher than TCP001-C.

3. The Stability of BCMA-TCPs in Human Plasma In Vitro

TCP001C, TCP002C and TCP003C were incubated in 100% human plasma at 37° C. for up to 21 days, and samples were respectively collected at Day 0, 1, 3, 7, 14, and 21. A 96-well plate was coated with mesothelin antigen, and after plate blocking and washing, collected samples with proper dilution together with serially diluted standard samples were incubated with the plate at 37° C. for 1 hr. Anti-VHH-cocktail-HRP (GenScript, A02016) was used as the detection antibody and the absorbance was read at 450 nm. Finally, the tested samples were analyzed according to the fitted curve of a standard sample group.

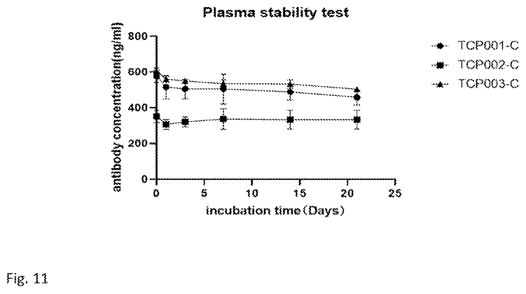

As shown in FIG. 11, TCP001-C, TCP002-C and TCP003-C are stable in human plasma in vitro at 37° C. for more than 21 days.

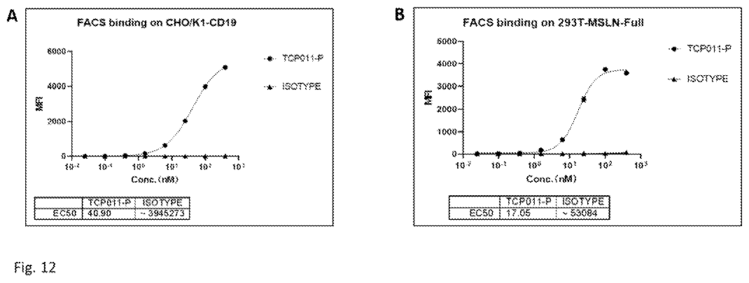

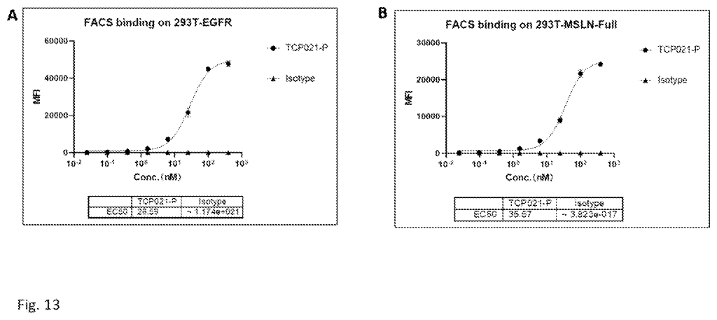

The binding affinity of TCP011-P for both CD19 and MSLN over-expressing cells (FIG. 12) as well as the binding affinity of TCP021-P for both the EGFR and MSLN over-expressing cells (FIG. 13) was confirmed by flow cytometry with the same procedure applied for the BCMA-TCPs.

### Example 8. In Vitro Amplification of M-ICAP-T with TCP Towards Target Cells

To verify the rapid activation and amplification of ICAP-T cells with TCP culturing with target cells, PBMC-T cells transfected with M-ICAP (activated by anti-His and anti-CD28) were co-cultured with CD19 positive Daudi lymphoma cells in the presence of TCP011-P or -N, respectively. CD19 positive Daudi lymphoma cells were treated with or without 50 ug/ml mitomycin C for 2 hr. 5×10⁵ PBMC-T cells transfected with M-ICAP cells were counted and cocultured with 5×10⁵ Daudi cells, as well as TCP011-P or -N for 4 days. The effector or target cells were then analyzed for proliferation by flow cytometry.

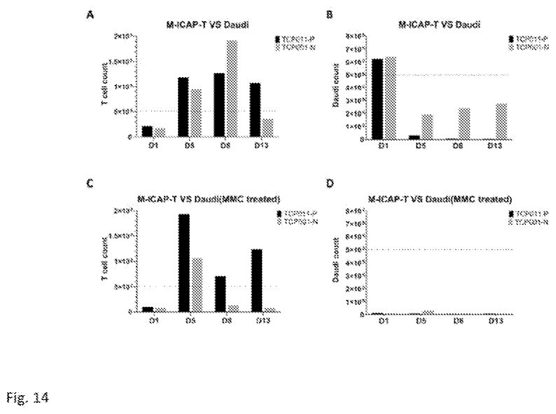

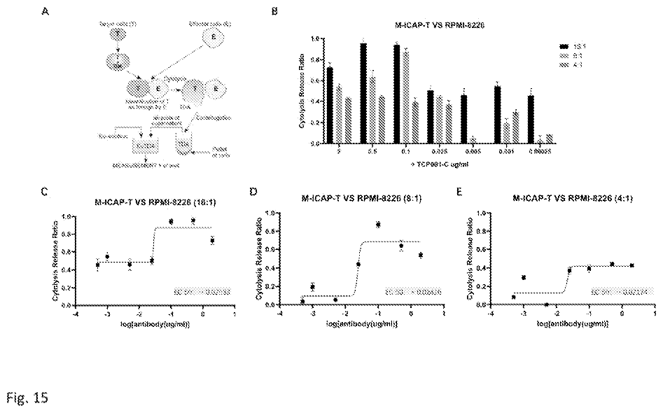

As shown in FIG. 14, in the presence of TCP011-P, M-ICAP-T cells transfected for 5, 8 and 13 days could effectively expand when stimulated by Daudi cells, amplification being highest at 5 days and 8 days after transfection. Further the activated M-ICAP-T cells could kill Daudi cells.

### Example 9. TCP-Dose-Dependent Cytotoxicity Effect of M-ICAP-T Towards RPMI-8226 Cells

In order to make ICAP-T cells act on BCMA positive tumor cells, it is necessary to have a TCP that can specifically bind to BCMA. The two ends of TCP001-C and -P can specifically bind ICAP-T and BCMA cells at the same time. In order to verify that, combined with specific TCP, ICAP-T can act on BCMA positive tumor cells and have specific cytolysis/killing effect, we compared the cytolysis/killing effect of ICAP-T or CAR-T cells co-cultured with RPMI-8226 or L363 cells (in three different E:T ratio) in the presence of different TCPs.

The cytotoxicity assay of T cells for suspension cell lines was conducted according to the manufacturer's protocol (DELFIA® EuTDA Cytotoxicity Reagents AD0116—PerkinElmer). Briefly, target tumor cells were washed with PBS and fluorescence enhancing ligand and incubated for 15 minutes at 37° C. 50 ul of target cells (5,000 cells) were placed into a V-bottom plate containing bispecific polypeptide that specifically binds to both of the target tumor cells and to the effector cells (that is, the transformed T cells), and 50 ul of effector cells are added with varying cell concentration (E:T=16/8/4:1). After 3.5 hours' incubation, 10 ul of the supernatant were transferred into 100 μL of Europium Solution. After 15 minutes incubation at room temperature, the fluorescence was measured in the time-resolved fluorometer. Specific release (%)=Experimental release (counts)−Spontaneous release (counts)/Maximum release (counts)−Spontaneous release (counts)×100.

IFNγ secretion by the T cells was also assayed. The IFNγ detection was conducted according to the manufacturer's protocol (IFNγ detection kit, VAL104—Novus). Briefly, fresh washing solution, colorant, diluent and standard product were prepared according to instructions. Different concentrations of standards and diluted experimental samples were added into the corresponding wells for 100 ul/well. The reaction well was sealed with sealing tape and incubated at room temperature for 2 hours. After 4 times washing using wash-buffer, 200 uL of enzyme labeled antibody was added into each well for 2 hours incubation at room temperature. After repeating the plate washing operation, 200 uL of previously mixed color reagent is added to each well, and the reaction incubated for 10-30 min in the dark. The color of the solution will change from blue to yellow by adding 50 ul/well termination solution. OD values are recorded with a spectrophotometer in 20 minutes and analyzed in Excel using a selected “four parameter equation” to obtain standard curve using a standard sample group.

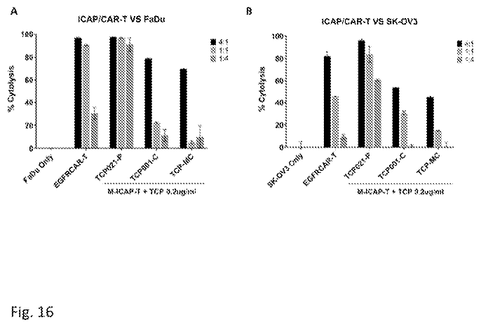

As shown in FIG. 16, M-ICAP-T combined with TCP001-C had strong specific killing effect on tumor target cells, however the nonspecific killing effect of T cells was more obvious when E:T=16:1. When the TCP concentration is >0.025 ug/ml, the cytolysis effects of E:T=8:1 and 4:1 are increasing. When the concentration of TCP001-C is 0.1 and 0.5 ug/ml, E:T=16:1 and 8:1 showed the best killing effect. When the concentration of TCP001-C reaches 2 ug/ml, the effect of E:T=16:1 and 8:1 decreases instead. E:T=16:1 showed the best killing effect. Under different E:T ratios, EC50 values of TCPs were similar (EC50=0.028 (E:T=16:1), 0.024 (E:T=8:1), 0.022 (E:T=4:1)), but the maximum value was corresponding to the E:T ratios.

### Example 10. Cytotoxicity Comparison and IFNγ Secreting Detection of ICAP-T Combined with Different TCPs Towards RPMI-8226/L363 Cells

The two ends of TCP001-C/P, TCP002-C/P and TCP003-C/P bind M-ICAP and BCMA at the same time. To verify and compare the specific cytolysis effect of ICAP-T cells combined with these TCPs on BCMA positive tumor cells, a cytolysis/killing assay of ICAP-T or CAR-T cells co-cultured with RPMI-8226 or L363 cells (E:T=8:1) was performed in the presence of various TCPs. The cytotoxicity and the IFNγ secretion assays of T cells for suspension cell lines are described in Example 9.

As shown in the FIG. 16, M-ICAP-T combined with TCP001-C has strong specific killing effect on tumor target cells, while the combination of TCP001-C(binding to BCMA positive cells only) or TCP-MD (no binding to BCMA positive cells) cannot effectively kill FaDu/SK-OV3 cells. Furthermore, the IFNγ secretion data are consistent with the cytotoxicity data. M-ICAP-T combined with both 0.2 ug/ml TCP001-C/P specifically induced IFNγ release on RPMI-8226 and L363 cell lines, comparing to the negative control groups (TCP001-N or TCP-MD or IgG). The rank of IFNγ release is: TCP001>TCP003>TCP002.

### Example 11. Cytolysis Effect of ICAP-T Cells Combined with TCP (Binding EGFR) on FaDu/SK-OV3 Cells

In order for ICAP-T cells to act on EGFR positive tumor cells, it is necessary to have a TCP that can specifically bind to EGFR. TCP021-P can combine ICAP and EGFR at its two ends, respectively. To verify that ICAP-T cells combined with this specific TCP can act on EGFR positive tumor cells such as FaDu (human pharyngeal squamous cell carcinoma) and SK-OV3 (human ovarian cancer cells), we compared the killing effect of ICAP-T or CAR-T cells co-cultured with FaDu/SK-OV3 cells in the presence of various TCPs.

The cytotoxicity assay of T cells for adhesion cell lines is conducted using an impedance-based RTCA TP instrument and method (xCELLigence). Target tumor cells are seeded in a resistor-bottomed 96-well plate at 10,000 cells per well within the RTCA TP instrument overnight (more than 16 hours). The bispecific TCP or antibodies are added to the cultured target tumor cells and the cells are further cultured for 30 minutes. Then ICAP-T or CAR-T cells are incubated with target tumor cells at different effector cell:target cell ratios for about 100 hours (the end point depends on the killing efficiency of transformed T cells). During the experiment, the cell index values are closely correlated with tumor cell adherence, such that lower cell attachment indicates higher cytotoxicity, and are collected every 5-10 minutes by the RTCA system. The real-time killing curves are automatically generated by the system software. Specific lysis (%) of each transformed T cell are also calculated using the data of the 48 h point [specific lysis=(cell index of tumor cells alone−cell index of transformed T cells cocultured with tumor cells)/cell index of tumor cells alone].

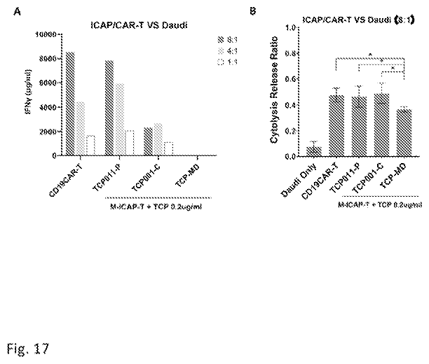

As shown in FIG. 17, similar to EGFRCAR-T, M-ICAP-T combined with TCP021-P has strong specific killing effect on the two different tumor target cells (the nonspecific killing effect of T cells is more obvious on E:T=4:1), while the combination of TCP001-C(binding BCMA positive cells only) or TCP-MD (no binding EGFR positive cells) cannot kill FaDu or SK-OV3 cells effectively.

### Example 12. IFN-γ Release and Cytolysis Effect of ICAP-T Cells with TCPs Towards Daudi Cells

In order for ICAP-T cells to act on B-Cells, it is necessary to have TCP that can specifically bind to CD19. TCP011-P can bind ICAP and CD19 at its two ends, respectively. To verify the effect of ICAP-T cells combined with this specific TCP on CD19 positive B cells, we compared the cytolysis of Daudi cells and the release of IFN-γ when ICAP-T or CAR-T cells were co-cultured with Daudi cells in the presence of various TCPs. The cytotoxicity and the IFNγ secreting detection assay of T cells for suspension cell lines are as described in Example 9.

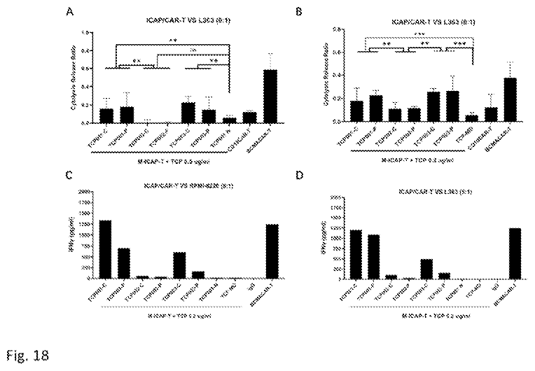

As shown in the FIG. 18, IFN-γ secretion shows significant difference. M-ICAP-T combined with TCP011-P was similar to CD19CAR-T in killing and IFN-γ secretion. Although TCP001-C(binds to BCMA) developed killing effect on Daudi cells, the level of IFN-γ secreted by T cells decreased significantly. Also, IFN-γ could not be secreted effectively when combined with TCP-MD (unable to bind Daudi cells).

### Example 13. Generation and Characterization of VHH Secreting ICAP-T

Due to complicated tumor microenvironment, most CAR-T therapies targeting solid tumors tested clinically at present show little clinical efficacy. To enhance anti-tumor effects of the ICAP-T cell system, M-ICAP-T cells secreting an immune checkpoint inhibitor, for example anti-PD-1, an antagonist of suppression cytokines in tumor, such as anti-TGFβ, or the like, were produced by simultaneously transfecting human naïve T cells with M-ICAP peptide (from MII+III peptide) and a plasmid encoding the secreted immune checkpoint inhibitor.

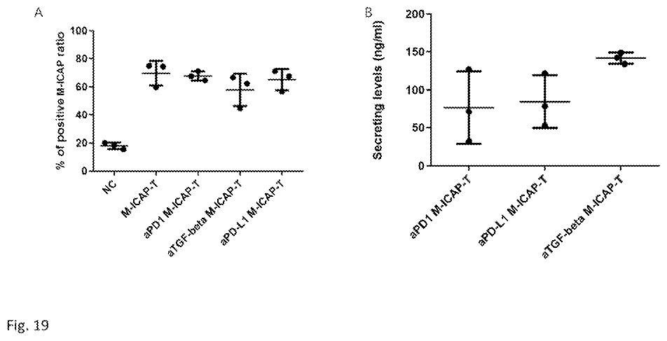

After 13 days of M-ICAP-T preparation, FACS was used for ICAP expression detection. Results are shown in FIG. 19. Compared with M-ICAP-T control, the nature of the protein to be secreted as being VHH or scFv did not exert any clear effect on ICAP expression.

ELISA was used to test concentrations of secreting proteins. Supernatants were added to antigen-coated 96-well plates, HRP conjugated anti-VHH and HRP-anti-His were used for anti-PD-1 VHH, anti-PD-L1 VHH and anti-TGFβ scFv detection respectively. Results were shown in FIG. 19B, concentrations of secreted VHH were about 75 ng/ml, and of secreted anti-TGFβ scFv were about 150 ng/ml.

### Example 14. Secreting Anti-PD-1 VHH Blocked Surface Expressed PD-1

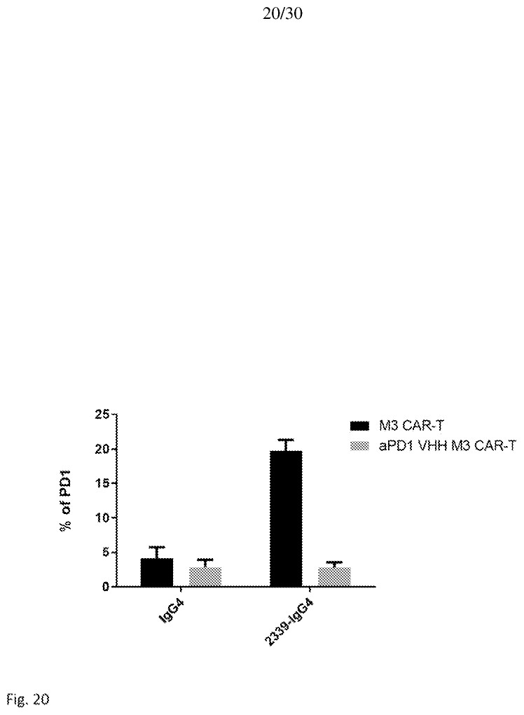

The binding ability of secreted anti-PD-1 VHH was tested indirectly by FACS of T cells expressing PD-1 on their surface. A commercially available anti-PD-1 antibody was used to examine PD-1 expression level on T cells in a competition assay. As FIG. 20 shows, supernatant from ICAP-T cells secreting anti-PD-1 blocked binding of a commercial anti-PD-1 antibody to CD3 and CD28 stimulated human primary T cells.

### Example 15. Anti-TGFβ scFv Secreted by M-ICAP-T Cells Blocked Luciferase Cell Signaling Induced by TGFβ-1

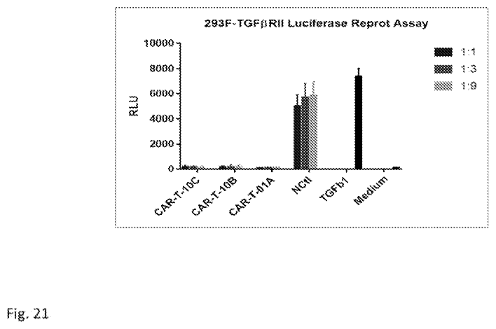

A commercially available TGFβRII-293T-Luc cell line was used to determine blocking activities of secreted anti-TGFβ scFv obtained in Example 13. 5000 TGFβRII-293 cells were seeded and incubated overnight, test samples were added, followed by 5 nM TGFβ, after 6 hr, ONE-GLO was used to read bioluminescence. Results are shown in FIG. 21. Anti-TGFβ scFvs secreted by M-ICAP T cells blocked luciferase signals induced by TGFβ. CAR-T-10C, 10B and 01A are anti-TGFβ M-ICAP-T cells which were prepared from different donors.

### Example 16. In Vivo Efficacy Study of M-ICAP-T Used with TCP001-C in an L363-PDL1-LUC Orthotopic Tumor Model

The orthotopic tumor model experiment described below utilizes NPSG mice (NOD-Prkdc^{scid} IL2rg^{tm1}/Pnk).

1. Tumor Inoculation, Grouping, Drug Administration and Animal Observations
(a) L363-PDL1-luc tumor cells were cultured in RPMI-1640 medium supplemented with 10% heat inactivated fetal bovine serum, 100 U/ml penicillin and 100 μg/ml streptomycin at 37° C. in an atmosphere of 5% CO2 in air. The cells growing in an exponential growth phase were harvested and counted for tumor inoculation. Cell count at the time of inoculation was 4.09E+8; viability was 83.65%.
(b) For the efficacy study, each mouse was inoculated IV with 2*10⁶ L363-PDL1-luc cells in 200 uL PBS. The date of tumor inoculation was defined as Day 0. When the tumor volume reached approximately 9.4E5 at Day 8, 24 mice were selected and randomly grouped into seven groups according to the animal body weight and tumor volume. Each group has 3-5 tumor-bearing mice. Seven groups were set based on the categories of drugs administered, dosage and dosing frequency. Group1 was set as the negative control group with injection of PBS only in the whole trial phase. Group 2 was another negative control group which was injected with high dose (20*E6) of anti-PD-1 M-ICAP-T cells intravenously at Day 8 and thereafter with PBS every 2 days for 7 times subcutaneously. Group3 was the positive control group which was injected with 5*E6 classic BCMA CAR-T(B2121) intravenously at Day 8 and thereafter with PBS every 2 days for 7 times subcutaneously. Group 4 and Group 5 were two experimental groups which were respectively injected with low dose (5*E6) and high dose (20*E6) anti-PD-1 M-ICAP-T cells and thereafter with 5 mg/kg TCP001-C every 2 days for 7 times subcutaneously. Group 6 and Group 7 were another two experimental groups which were respectively injected with 5*E6 and 20*E6 M-ICAP-T cells and thereafter with 5 mg/kg TCP001-C every 2 days for 7 times subcutaneously.
(c) All the procedures related to animal handling, care and the treatment in this study were performed according to the guidelines approved by the Institutional Animal Care and Use Committee (IACUC) of BioDuro following the guidance of the Association for Assessment and Accreditation of Laboratory Animal Care (AAALAC, accreditation number is 001516. At the time of routine monitoring, the animals were checked for any adverse effects of tumor growth and/or treatment on normal behavior such as effects on mobility, food and water consumption (by observation only), and body weight gain/loss (body weights were measured twice weekly in the pre-dosing phase and daily in the dosing phase), eye/hair matting and any other abnormal effect, including tumor ulceration. The sponsor was informed when the body weight loss of any animal reached 10%.

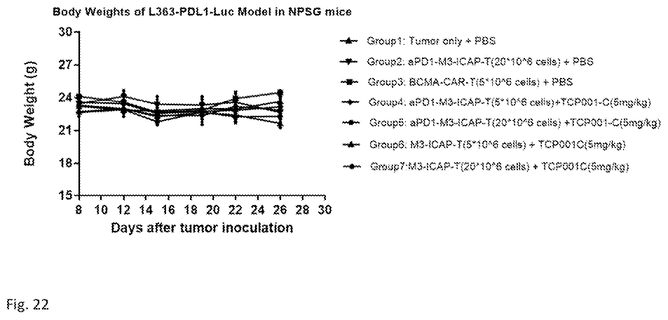

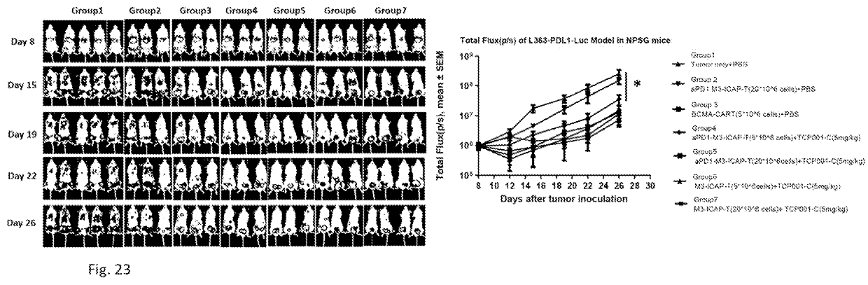

2. Body Weight; Tumor Measurements
Body weight and bioluminescence signals were measured twice a week. Results of the body weight changes in the tumor bearing mice are shown in FIG. 22. No abnormal body weight changes were observed in any of the groups during the trial.
Bioluminescence signals in mice were measured twice a week using the IVIS lumina XR, starting from Day 4 post cell injection and throughout the study. The signals were quantified by the Living Image software. As shown in FIG. 23, during the study period (Day 8-Day 26), there is no significant difference in efficacy between Group 2 (injected with M-ICAP-T only after tumor inoculation) and Group 1 (tumor inoculation only). In comparison, all the experimental groups (Groups 4, 5, 6 and 7), which were injected with M-ICAP-T activated by regular TCP001-C showed significant efficacy against L363-PDL1 in the orthotopic tumor model compared to the two negative control groups (Group 1 and Group 2). Furthermore, the experiment groups (Groups 4, 5, 6 and 7) showed similar efficacy to the classic BCMA CAR-T therapy (Group 3).
Injection of M-ICAP-T cells together with regular TCP001-C injection at the frequency of once every two days can significantly suppress tumors in the established L363-PDL1-LUC orthotopic tumor model.

3. Analysis of Anti-PD-1 and TCP001-C Concentration in Whole Blood of Mice

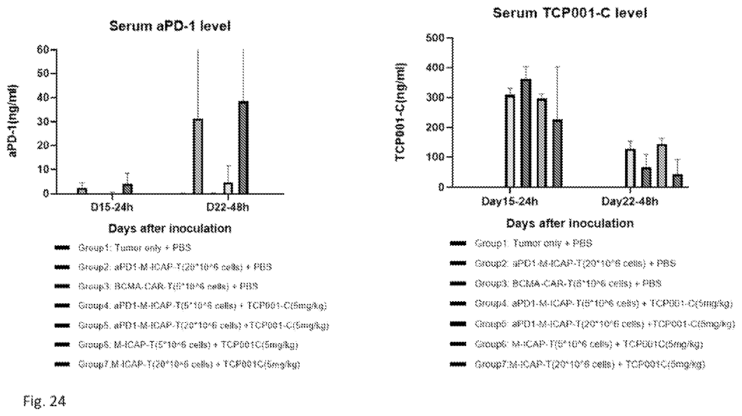

100 μl of peripheral blood was collected once a week for analysis of the count of the anti-PD-1 and TCP001-C concentration in whole blood of mice. 1 μg/ml PD-1 protein was coated on 96-well plates overnight for ELISA binding assay. Diluted samples together with diluted standard samples (8 dilution points from 2 ng/ml) were added to the wells and let stand for 1 hr at 37° C. Then anti VHH-cocktail antibody was added as detection antibody and the assay reagents are added. The absorbance was read at 450 nm. Concentrations were determined by analysis against the standard curve as in Example 9.
As shown in FIG. 24, anti-PD-1 VHH can only be significantly detected in the Groups 2, 4 and 5 at two sampling time points (Day 15, Day 22). Groups 2, 4 and 5 used anti-PD-1 M-ICAP-T as the effector T cells, indicating that PD-1 can be successfully secreted by the anti-PD-1 M-ICAP-T cells in vivo.
The method for analysis of TCP001-C concentration has been described in the method “Stability evaluation for Antibodies in human plasma” in the antibody production and characterization part (Example 5). High concentration of TCP001-C can be detected in the peripheral blood sampled at 24 hr. as well as 48 hr. after TCP001-C subcutaneous injection. This indicates that the half-life of TCP001-C in vivo is more than 48 hr and the injection frequency of once every two days is sufficient to support the efficacy of M-ICAP-T towards L363 tumor cells.

### Example 17: Identification and Characterization of an Exemplary BCMA Peptide Motif as a Universal Tagging System for In Vitro and In Vivo Amplification of CAR-T

A peptide motif (about 20-30 aa in length) fused to the N terminal of the antigen binding domain (scFv or VHH) of a CAR-T cell receptor as a universal ICAP for CAR-T amplification in vitro or in vivo. The following criteria were used for the peptide motif design.

First, the peptide is about 20-30 aa in length. Second, nanobodies specific to this peptide motif with high binding affinity (KD<1 nM) can be obtained. Finally, the nanobodies targeting the peptide successfully induced CAR-T in vitro or in vivo amplification when the peptide was fused to the N terminal of the antigen binding domain (scFv or VHH) of the chimeric antigen receptor of CAR-T cells.

1. Identification and Characterization of One VHH Sequence with High Affinity Towards MSLN.

We identified one VHH nanobody towards MSLN with high affinity using an alpaca immune library. The procedures screening for alpaca immunization, blood collection, library construction, solid panning, ELISA or FACS screening for positive clones, antibody purification and followed antibody characterization by SPR and FACS were as described in Example 2. One positive clone named as anti-MSLN-1444 VHH was obtained.

As shown in Table 6, the affinity of anti-MSLN-1444 VHH towards MSLN His antigen was high with KD of 2.10E-09.

TABLE 6
Kinetics of binding of BCMA-TCPs to BMC A

| Antibody | Kon (1/Ms) | Koff(1/s) | KD(M) |
|---|---|---|---|
| 1444(VHH) | 5.37E+04 | 7.50E−05 | 2.10E−09 |

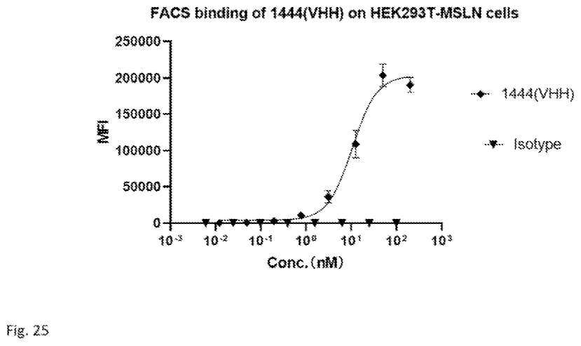

As shown in FIG. 25, anti-MSLN-1444 VHH showed good and specific binding affinity towards HEK293T-MSLN cells.

2. Identification of BCMA Peptide (BCMA ICAP) which can be Recognized Potently by BCMA Full Length VHH Binders.

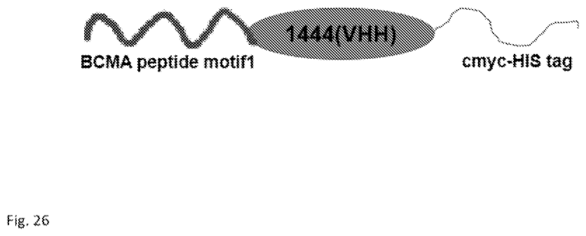

One BCMA peptide motif with proper length (˜20 aa) that can be recognized with high affinity by some BCMA candidate binding proteins from a previously prepared immune library with BCMA-hFc antigen and many candidate VHH sequences with various binding properties to full length BCMA was designed. One polypeptide BCMA mut1 from natural BCMA (1-23aa of BCMA ECD domain, Table 7—SEQ ID NO: 17) was selected, and a fusion polypeptide of BCMAmut1 and anti-MSLN-1444 VHH sequence targeting MSLN to be expressed is shown in FIG. 26.

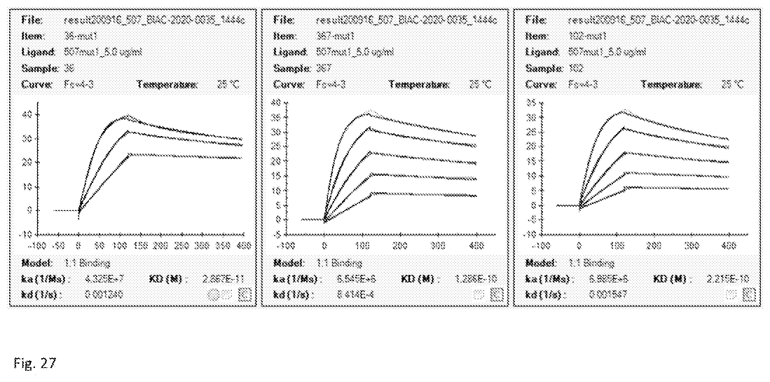

Then we filtered out three VHH sequences (#36, #102 and #367 described below) with high affinity to it. The sequence of BCMA mut1 is also shown in Table 7 (SEQ ID NO:18), The three VHH sequences were expressed as a hFc fusion proteins, named as 36(VHH), 102(VHH) and 367(VHH) with the procedure that was described in the patent publication WO2020176815A2 (hereby incorporated by reference in its entirety and for all purposes). The binding affinity of three VHH nanobodies was measured by SPR. As shown in FIG. 27, three VHHs showed high affinity binding to BCMAmut1. Binding kinetics parameters of these three VHHs are shown in Table 8. Anti-BCMA VHH 36# was selected as stimulator due to its higher affinity to BCMA ICAP BMCAmut1.

TABLE 7
Sequences of BCMA ICAP BCMAmut1.

| Protein/ | |
|---|---|
| peptide ID | Sequence |
| BCMA ECD | MLQMAGQCSQ NEYFDSLLHA CIPCQLRCSS |
| | NTPPLTCQRY CNASVTNSVK GTNA |
| | (SEQ ID NO: 17) |
| BCMA mut1 | MLQMAGQCSQ NEYFDSLLHA CIP |
| | (SEQ ID NO: 18) |

TABLE 8
Kinetic parameters of anti-BCMA VHHs to BCMA ICAP BCMAmut1

| Analyte Ligand | ka (1/Ms) | kd (1/s) | KD (M) |
|---|---|---|---|
| BCMA mut1 #36 | 4.33E+07 | 1.24E−03 | 2.87E−11 |
| BCMA mut1 #367 | 6.55E+06 | 8.41E−04 | 1.29E−10 |
| BCMA mut1 #102 | 6.99E+06 | 0.001547 | 2.22E−10 |

3. BCMA ICAP can be Used to Specifically Amplify CAR T Cells with BCMA ICAP.

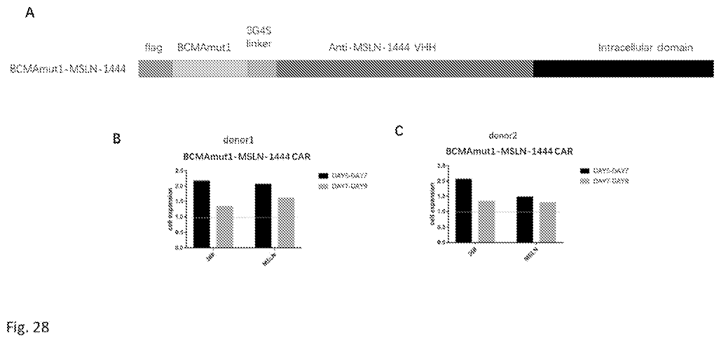

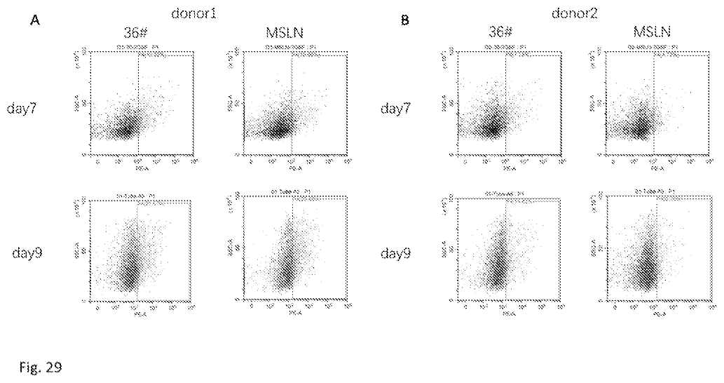

An ICAP-1-23-3GS vector was constructed as illustrated in FIG. 28A, with BCMAmut1 (ICAP) at the N terminal of anti-MSLN CAR connected with a (G4S)3 linker. BCMAmut1-MSLN-1444 CAR-T cells were prepared by transfection with the BCMAmut1-MSLN-1444 vector followed by stimulation with plated-coated MSLN and anti-CD28 or anti-BCMAmut1 36# and anti-CD28 respectively. CAR-T cells showed comparable amplification ability stimulated by anti-BCMAmut1 36# in 2 donors (FIGS. 28B, 28C and FIGS. 29A, 29B) compared with CAR-T cells stimulated by antigen MSLN after 9 days culture.

4. Anti BCMA Mut1 36# Did not Stimulate Non-Specific Amplification of CAR T Cells with BCMA ICAP.

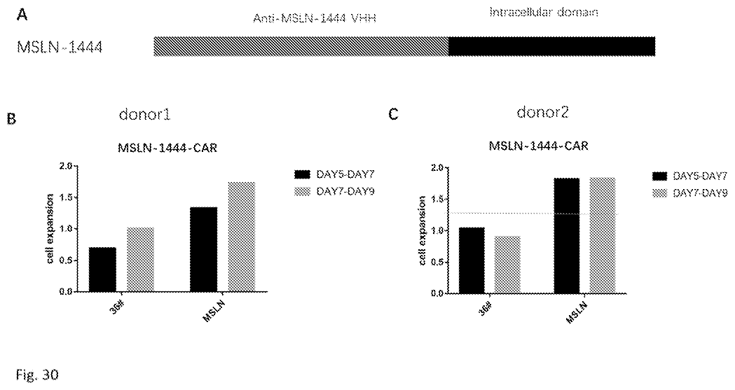

To test the specificity of stimulation of anti-BCMAmut1 36# to BCMA ICAP CAR-T cells, a MSLN-1444 CAR vector was constructed as shown in FIG. 30A, and were prepared by transfection of PBMC followed by stimulation with plated-coated MSLN and anti-CD28 or anti-BCMAmut1 36# and anti-CD28 respectively. Results are shown in FIG. 30B; only those CAR T cells stimulated with MSLN and anti-CD28 showed clear amplification. MSLN-1444 CAR stimulated with anti-BCMA 36# and anti-CD28 did not display amplification in 2 donors.

### ADDITIONAL NUCLEIC ACID AND AMINO ACID SEQUENCES

MSLN region II+ region III (M3) DNA:
(SEQ ID NO:19)
TCCCTGGAGACCCTGAAGGCTTTGCTTGAAGTCAACAAAGGGCACGAAA
TGAGTCCTCAGGTGGCCACCCTGATCGACCGCTTTGTGAAGGGAAGGGG
CCAGCTAGACAAAGACACCCTAGACACCCTGACCGCCTTCTACCCTGGG
TACCTGTGCTCCCTCAGCCCCGAGGAGCTGAGCTCCGTGCCCCCCAGCA
GCATCTGGGCGGTCAGGCCCCAGGACCTGGACACGTGTGACCCAAGGCA
GCTGGACGTCCTCTATCCCAAGGCCCGCCTTGCTTTCCAGAACATGAAC
GGGTCCGAATACTTCGTGAAGATCCAGTCCTTCCTGGGTGGGGCCCCCA
CGGAGGATTTGAAGGCGCTCAGTCAGCAGAATGTGAGCATGGACTTGGC
CACGTTCATGAAGCTGCGGACGGATGCGGTGCTGCCGTTGACTGTGGCT
GAGGTGCAGAAACTTCTGGGACCCCACGTGGAGGGCCTGAAGGCGGAGG
AGCGGCACCGCCCGGTGCGGGACTGGATCCTACGGCAGCGGCAGGACGA
CCTGGACACGCTGGGGCTGGGGCTACAGGGCGGCATCCCCAACGGCTAC
CTGGTCCTAGACCTCAGCATGCAAGAGGCCCTCTCG
MSLN region II+ region III (M3) protein:
(SEQ ID NO:20)
SLETLKALLEVNKGHEMSPQVATLIDRFVKGRGQLDKDTLDTLTAFYPG
YLCSLSPEELSSVPPSSIWAVRPQDLDTCDPRQLDVLYPKARLAFQNMN
GSEYFVKIQSFLGGAPTEDLKALSQQNVSMDLATFMKLRTDAVLPLTVA
EVQKLLGPHVEGLKAEERHRPVRDWILRQRQDDLDTLGLGLQGGIPNGY
LVLDLSMQEALS
M-ICAP CAR ORF DNA:
(SEQ ID NO:21)
ATGGCCTTGCCAACGGCTCGACCCCTGTTGGGGTCCTGTGGGACCCCCG
CCCTCGGCAGCCTCCTGTTCCTGCTCTTCAGCCTCGGATGGGTGCAGCC
CCACCACCACCATCACCACGGAGGAGGCGGATCTTCCCTGGAGACCCTG
AAGGCTTTGCTTGAAGTCAACAAAGGGCACGAAATGAGTCCTCAGGTGG
CCACCCTGATCGACCGCTTTGTGAAGGGAAGGGGCCAGCTAGACAAAGA
CACCCTAGACACCCTGACCGCCTTCTACCCTGGGTACCTGTGCTCCCTC
AGCCCCGAGGAGCTGAGCTCCGTGCCCCCCAGCAGCATCTGGGCGGTCA
GGCCCCAGGACCTGGACACGTGTGACCCAAGGCAGCTGGACGTCCTCTA
TCCCAAGGCCCGCCTTGCTTTCCAGAACATGAACGGGTCCGAATACTTC
GTGAAGATCCAGTCCTTCCTGGGTGGGGCCCCCACGGAGGATTTGAAGG
CGCTCAGTCAGCAGAATGTGAGCATGGACTTGGCCACGTTCATGAAGCT
GCGGACGGATGCGGTGCTGCCGTTGACTGTGGCTGAGGTGCAGAAACTT
CTGGGACCCCACGTGGAGGGCCTGAAGGCGGAGGAGCGGCACCGCCCGG
TGCGGGACTGGATCCTACGGCAGCGGCAGGACGACCTGGACACGCTGGG
GCTGGGGCTACAGGGCGGCATCCCCAACGGCTACCTGGTCCTAGACCTC
AGCATGCAAGAGGCCCTCTCGATCTACATCTGGGCGCCCCTGGCCGGGA
CTTGTGGGGTCCTTCTCCTGTCACTGGTTATCACCCTTTACTGCAAACG
GGGCAGAAAGAAGCTCCTGTATATATTCAAACAACCATTTATGAGACCA
GTACAAACTACTCAAGAGGAAGATGGCTGTAGCTGCCGATTTCCAGAAG
AAGAAGAAGGAGGATGTGAACTGAGAGTGAAGTTCAGCAGGAGCGCAGA
CGCCCCCGCGTACCAGCAGGGCCAGAACCAGCTCTATAACGAGCTCAAT
CTAGGACGAAGAGAGGAGTACGATGTTTTGGACAAGAGACGTGGCCGGG
ACCCTGAGATGGGGGGAAAGCCGAGAAGGAAGAACCCTCAGGAAGGCCT
GTACAATGAACTGCAGAAAGATAAGATGGCGGAGGCCTACAGTGAGATT
GGGATGAAAGGCGAGCGCCGGAGGGGCAAGGGGCACGATGGCCTTTACC
AGGGTCTCAGTACAGCCACCAAGGACACCTACGACGCCCTTCACATGCA
GGCCCTGCCCCCTCGCTGA
M-ICAP CAR ORF protein:
(SEQ ID NO:22)
MALPTARPLLGSCGTPALGSLLFLLFSLGWVQPHHHHHHGGGGSSLETL
KALLEVNKGHEMSPQVATLIDRFVKGRGQLDKDTLDTLTAFYPGYLCSL
SPEELSSVPPSSIWAVRPQDLDTCDPRQLDVLYPKARLAFQNMNGSEYF
VKIQSFLGGAPTEDLKALSQQNVSMDLATFMKLRTDAVLPLTVAEVQKL
LGPHVEGLKAEERHRPVRDWILRQRQDDLDTLGLGLQGGIPNGYLVLDL
SMQEALSIYIWAPLAGTCGVLLLSLVITLYCKRGRKKLLYIFKQPFMRP
VQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEI
GMKGERRRGKGHDGLYQGLSTATKDTYDALHMQALPPR
M-ICAP-SP3 CAR ORF DNA:
(SEQ ID NO:23)
ATGAAGCACCTCTGGTTCTTCCTCCTGCTGGTGGCAGCTCCTAGATGGG
TGCTGTCTCACCACCACCATCACCACGGAGGAGGCGGATCTTCCCTGGA
GACCCTGAAGGCTTTGCTTGAAGTCAACAAAGGGCACGAAATGAGTCCT
CAGGTGGCCACCCTGATCGACCGCTTTGTGAAGGGAAGGGGCCAGCTAG
ACAAAGACACCCTAGACACCCTGACCGCCTTCTACCCTGGGTACCTGTG
CTCCCTCAGCCCCGAGGAGCTGAGCTCCGTGCCCCCCAGCAGCATCTGG
GCGGTCAGGCCCCAGGACCTGGACACGTGTGACCCAAGGCAGCTGGACG
TCCTCTATCCCAAGGCCCGCCTTGCTTTCCAGAACATGAACGGGTCCGA
ATACTTCGTGAAGATCCAGTCCTTCCTGGGTGGGGCCCCCACGGAGGAT
TTGAAGGCGCTCAGTCAGCAGAATGTGAGCATGGACTTGGCCACGTTCA
TGAAGCTGCGGACGGATGCGGTGCTGCCGTTGACTGTGGCTGAGGTGCA
GAAACTTCTGGGACCCCACGTGGAGGGCCTGAAGGCGGAGGAGCGGCAC
CGCCCGGTGCGGGACTGGATCCTACGGCAGCGGCAGGACGACCTGGACA
CGCTGGGGCTGGGGCTACAGGGCGGCATCCCCAACGGCTACCTGGTCCT
AGACCTCAGCATGCAAGAGGCCCTCTCGATCTACATCTGGGCGCCCCTG
GCCGGGACTTGTGGGGTCCTTCTCCTGTCACTGGTTATCACCCTTTACT
GCAAACGGGGCAGAAAGAAGCTCCTGTATATATTCAAACAACCATTTAT
GAGACCAGTACAAACTACTCAAGAGGAAGATGGCTGTAGCTGCCGATTT
CCAGAAGAAGAAGAAGGAGGATGTGAACTGAGAGTGAAGTTCAGCAGGA
GCGCAGACGCCCCCGCGTACCAGCAGGGCCAGAACCAGCTCTATAACGA
GCTCAATCTAGGACGAAGAGAGGAGTACGATGTTTTGGACAAGAGACGT
GGCCGGGACCCTGAGATGGGGGGAAAGCCGAGAAGGAAGAACCCTCAGG
AAGGCCTGTACAATGAACTGCAGAAAGATAAGATGGCGGAGGCCTACAG
TGAGATTGGGATGAAAGGCGAGCGCCGGAGGGGCAAGGGGCACGATGGC
CTTTACCAGGGTCTCAGTACAGCCACCAAGGACACCTACGACGCCCTTC
ACATGCAGGCCCTGCCCCCTCGCTGA
M-ICAP-SP3 CAR ORF protein:
(SEQ ID NO:24)
MKHLWFFLLLVAAPRWVLSHHHHHHGGGGSSLETLKALLEVNKGHEMSP
QVATLIDRFVKGRGQLDKDTLDTLTAFYPGYLCSLSPEELSSVPPSSIW
AVRPQDLDTCDPRQLDVLYPKARLAFQNMNGSEYFVKIQSFLGGAPTED
LKALSQQNVSMDLATFMKLRTDAVLPLTVAEVQKLLGPHVEGLKAEERH
RPVRDWILRQRQDDLDTLGLGLQGGIPNGYLVLDLSMQEALSIYIWAPL
AGTCGVLLLSLVITLYCKRGRKKLLYIFKQPFMRPVQTTQEEDGCSCRF
PEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDG
LYQGLSTATKDTYDALHMQALPPR
M-ICAP-SP5 CAR ORF DNA:
(SEQ ID NO:25)
ATGACCAGGCTGACAGTGCTGGCTCTGCTGGCCGGACTGCTGGCTTCTT
CTAGAGCTCACCACCACCATCACCACGGAGGAGGCGGATCTTCCCTGGA
GACCCTGAAGGCTTTGCTTGAAGTCAACAAAGGGCACGAAATGAGTCCT
CAGGTGGCCACCCTGATCGACCGCTTTGTGAAGGGAAGGGGCCAGCTAG
ACAAAGACACCCTAGACACCCTGACCGCCTTCTACCCTGGGTACCTGTG
CTCCCTCAGCCCCGAGGAGCTGAGCTCCGTGCCCCCCAGCAGCATCTGG
GCGGTCAGGCCCCAGGACCTGGACACGTGTGACCCAAGGCAGCTGGACG
TCCTCTATCCCAAGGCCCGCCTTGCTTTCCAGAACATGAACGGGTCCGA
ATACTTCGTGAAGATCCAGTCCTTCCTGGGTGGGGCCCCCACGGAGGAT
TTGAAGGCGCTCAGTCAGCAGAATGTGAGCATGGACTTGGCCACGTTCA
TGAAGCTGCGGACGGATGCGGTGCTGCCGTTGACTGTGGCTGAGGTGCA
GAAACTTCTGGGACCCCACGTGGAGGGCCTGAAGGCGGAGGAGCGGCAC
CGCCCGGTGCGGGACTGGATCCTACGGCAGCGGCAGGACGACCTGGACA
CGCTGGGGCTGGGGCTACAGGGCGGCATCCCCAACGGCTACCTGGTCCT
AGACCTCAGCATGCAAGAGGCCCTCTCGATCTACATCTGGGCGCCCCTG
GCCGGGACTTGTGGGGTCCTTCTCCTGTCACTGGTTATCACCCTTTACT
GCAAACGGGGCAGAAAGAAGCTCCTGTATATATTCAAACAACCATTTAT
GAGACCAGTACAAACTACTCAAGAGGAAGATGGCTGTAGCTGCCGATTT
CCAGAAGAAGAAGAAGGAGGATGTGAACTGAGAGTGAAGTTCAGCAGGA
GCGCAGACGCCCCCGCGTACCAGCAGGGCCAGAACCAGCTCTATAACGA
GCTCAATCTAGGACGAAGAGAGGAGTACGATGTTTTGGACAAGAGACGT
GGCCGGGACCCTGAGATGGGGGGAAAGCCGAGAAGGAAGAACCCTCAGG
AAGGCCTGTACAATGAACTGCAGAAAGATAAGATGGCGGAGGCCTACAG
TGAGATTGGGATGAAAGGCGAGCGCCGGAGGGGCAAGGGGCACGATGGC
CTTTACCAGGGTCTCAGTACAGCCACCAAGGACACCTACGACGCCCTTC
ACATGCAGGCCCTGCCCCCTCGCTGA
M-ICAP-SP5 CAR ORF protein:
(SEQ ID NO:26)
MTRLTVLALLAGLLASSRAHHHHHHGGGGSSLETLKALLEVNKGHEMSP
QVATLIDRFVKGRGQLDKDTLDTLTAFYPGYLCSLSPEELSSVPPSSIW
AVRPQDLDTCDPRQLDVLYPKARLAFQNMNGSEYFVKIQSFLGGAPTED
LKALSQQNVSMDLATFMKLRTDAVLPLTVAEVQKLLGPHVEGLKAEERH
RPVRDWILRQRQDDLDTLGLGLQGGIPNGYLVLDLSMQEALSIYIWAPL
AGTCGVLLLSLVITLYCKRGRKKLLYIFKQPFMRPVQTTQEEDGCSCRF
PEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDG
LYQGLSTATKDTYDALHMQALPPR
M(2339VHH) DNA sequence:
(SEQ ID NO:27)
CAGCTGCAGCTGGGCGCCTCTGGCGGCGGCCTGGTCCAGCCTGGCGGCT
CTCTGAGACTGAGCTGTGCCCTGTCTGGCTTCACACTGAGAGAGCTGGA
CGAGTTCGCCATCGGCTGGTTCAGGCAGGCCCCTGGCAAGGAGAGAGAG
GGCGTGAGCTGTATCAGCGGCACAGGCGGCATCACACATTATGCTGACA
GCGTGAAGGGCAGGTTCACAATCAGCAGAGACATCGCCAAGACAACCGT
GTACCTGCAGATGAATAGCCTGAACAGCGAAGACACAGCCGTGTACTAC
TGTGCCGCCGACGAGAGATGTACAGACAGACTGATCAGACCTCCTACAT
ATTGGGGACAAGGCACCCAGGTGACAGTCTCTTCT
M(2339VHH) protein sequence:
(SEQ ID NO:28)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSS
BCMA B029(VHH) sequence in TCP001-C and MC001C:
(SEQ ID NO:29)
QVQLVESGGGLVQPGGSLRLSCAASGSITSIYAIGWYRQAPGKLRELVA
AITTSGNTFYRDSVKGRFTISRDNAKNTVSLQMNSLKSEDTAVYDCNGA
PWGDHAPLVVSWDQGTQVTVSS
CD19 scFv sequence (from CN201480027401.4.) in
TCP011-P:
(SEQ ID NO:30)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITKAGGGGSGGGGSGGGGSEVKLQESGPGLVAPSQSLSVTCT
VSGVSLPDYGVSWIRQPPRKGLEWLGVIWGSETTYYNSALKSRLTIIKD
NSKSQVFLKMNSLQTDDTAIYYCAKHYYYGGSYAMDYWGQGTSVTVSS
EGFR E454(VHH) sequence in TCP021-P:
(SEQ ID NO:31)
QVQLVESGGGLVQPGGSLNLSCAASGFDFSSVTMSWHRQSPGKERETVA
VISNIGNRNVGSSVRGRFTISRDNKKQTVHLQMDNLKPEDTGIYRCKAW
GLDLWGPGTQVTVSS
GFP scFv sequence in TCP001-N:
(SEQ ID NO:32)
QVQLVESGGALVQPGGSLRLSCAASGFPVNRYSMRWYRQAPGKEREWVA
GMSSAGDRSSYEDSVKGRFTISRDDARNTVYLQMNSLKPEDTAVYYCNV
NVGFEYWGQGTQVTVSS
Anti-TGFβ scF (mAb12.7) - from US7494651B2:
(SEQ ID NO:33)
QVQLVQSGAEVKKPGASVKVSCKASGYTFTSEWMNWVRQAPGQGLEWMG
QIFPALGSTNYNEMYEGRVTMTTDTSTSTAYMELRSLRSDDTAVYYCAR
GIGNYALDAMDYWGQGTLVTVSSGGGGSGGGGSGGGGSDIQMTQSPSSL
SASVGDRVTITCRASESVDFYGNSFMHWYQQKPGKAPKLLIYLASNLES
GVPSRFSGSGSGTDFTLTISSLQPEDFATYYCQQNIEDPLTFGGGTKVE
IK
PD-L1 BMK1 VHH (envafolimab) - from US20180327494:
(SEQ ID NO:34)
QVQLVESGGGLVQPGGSLRLSCAASGKMSSRRCMAWFRQAPGKERERVA
KLLTTSGSTYLADSVKGRFTISRDNSKNTVYLQMNSLRAEDTAVYYCAA
DSFEDPTCTLVTSSGAFQYWGQGTLVTVSS
1444(VHH) protein sequence:
(SEQ ID NO:35)
QVQVVESGGGFVQAGGSLRLSCAASTPIISIAYMGWYRQISEKERQLVA
TINSGGKTYYADSVKGRFTISRDNAKNTLYLQMNMLKPEDTGMYYCAAS
NKDYNDYDPDWGQGTQVTVSS
B2121 scFv sequence in TCP001-P:
(SEQ ID NO:36)
DIVLTQSPASLAMSLGERATISCRASESVSVIGAHLIHWYQQKPGQPPK
LLIYLASNLETGVPARFSGSGSGTDFTLTISRVQAEDAAIYSCLQSRIF
PRTFGQGTKLEIKGSTSGSGKPGSGEGSTKGQVQLVQSGSELKKPGESV
KISCKASGYTFTDYSINWVKQAPGQGLKWMGWINTETREPAYAYDFRGR
FVFSLDTSASTAYLQISSLKAEDTAVYFCALDYSYAMDYWGQGTLVTVS
S
TCP001-C:
(SEQ ID NO:37)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSGGGGSGGGGSQVQLVE
SGGGLVQPGGSLRLSCAASGSITSIYAIGWYRQAPGKLRELVAAITTSG
NTFYRDSVKGRFTISRDNAKNTVSLQMNSLKSEDTAVYDCNGAPWGDHA
PLVVSWDQGTQVTVSSGGGGSEQKLISEEDLGGGGSHHHHHH
TCP001-P:
(SEQ ID NO:38)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSGGGGSGGGGSDIVLTQ
SPASLAMSLGERATISCRASESVSVIGAHLIHWYQQKPGQPPKLLIYLA
SNLETGVPARFSGSGSGTDFTLTISRVQAEDAAIYSCLQSRIFPRTFGQ
GTKLEIKGSTSGSGKPGSGEGSTKGQVQLVQSGSELKKPGESVKISCKA
SGYTFTDYSINWVKQAPGQGLKWMGWINTETREPAYAYDFRGRFVFSLD
TSASTAYLQISSLKAEDTAVYFCALDYSYAMDYWGQGTLVTVSSGGGGS
EQKLISEEDLGGGGSHHHHHH
TCP001-N:
(SEQ ID NO:39)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSGGGGSGGGGSQVQLVE
SGGALVQPGGSLRLSCAASGFPVNRYSMRWYRQAPGKEREWVAGMSSAG
DRSSYEDSVKGRFTISRDDARNTVYLQMNSLKPEDTAVYYCNVNVGFEY
WGQGTQVTVSSGGGGSEQKLISEEDLGGGGSHHHHHH
TCP011-P:
(SEQ ID NO:40)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSGGGGSGGGGSDIQMTQ
TTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKL
EITKAGGGGSGGGGSGGGGSEVKLQESGPGLVAPSQSLSVTCTVSGVSL
PDYGVSWIRQPPRKGLEWLGVIWGSETTYYNSALKSRLTIIKDNSKSQV
FLKMNSLQTDDTAIYYCAKHYYYGGSYAMDYWGQGTSVTVSSGGGGSEQ
KLISEEDLGGGGSHHHHHH
TCP021-P:
(SEQ ID NO:41)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSGGGGSGGGGSQVQLVE
SGGGLVQPGGSLNLSCAASGFDFSSVTMSWHRQSPGKERETVAVISNIG
NRNVGSSVRGRFTISRDNKKQTVHLQMDNLKPEDTGIYRCKAWGLDLWG
PGTQVTVSSGGGGSEQKLISEEDLGGGGSHHHHHH
TCP002-C:
(SEQ ID NO:42)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSAAAESKYGPPCPPCPA
PEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSV
MHEALHNHYTQKSLSLSLGKGGGGSGGGGSGGGGSQVQLVESGGGLVQP
GGSLRLSCAASGSITSIYAIGWYRQAPGKLRELVAAITTSGNTFYRDSV
KGRFTISRDNAKNTVSLQMNSLKSEDTAVYDCNGAPWGDHAPLVVSWDQ
GTQVTVSS
TCP003-C:
(SEQ ID NO:43)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSAAAGQPREPQVYTLPP
SQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKGGGG
SGGGGSGGGGSQVQLVESGGGLVQPGGSLRLSCAASGSITSIYAIGWYR
QAPGKLRELVAAITTSGNTFYRDSVKGRFTISRDNAKNTVSLQMNSLKS
EDTAVYDCNGAPWGDHAPLVVSWDQGTQVTVSSGGGGSEQKLISEEDLG
GGGSHHHHHH
TCP-MC:
(SEQ ID NO:44)
QVQLVESGGGLVQPGGSLRLSCAASGSITSIYAIGWYRQAPGKLRELVA
AITTSGNTFYRDSVKGRFTISRDNAKNTVSLQMNSLKSEDTAVYDCNGA
PWGDHAPLVVSWDQGTQVTVSSGGGGSEQKLISEEDLGGGGSHHHHHH
TCP-MD:
(SEQ ID NO:45)
QLQLGASGGGLVQPGGSLRLSCALSGFTLRELDEFAIGWFRQAPGKERE
GVSCISGTGGITHYADSVKGRFTISRDIAKTTVYLQMNSLNSEDTAVYY
CAADERCTDRLIRPPTYWGQGTQVTVSSGGGGSEQKLISEEDLGGGGSH
HHHHH

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

### Embodiments

Embodiment 1: An immune cell that comprises an expressed immune cell activator polypeptide comprising an intracellular signal transduction domain, a transmembrane domain, and an extracellular label domain, wherein the immune cell secretes one or more polypeptide effector molecules.

Embodiment 2: An immune cell that comprises an expressed immune cell activator polypeptide comprising an intracellular signal transduction domain, a transmembrane domain, and an extracellular chimeric polypeptide comprising a binding domain of a VHH antibody or a single chain variable fragment and a label domain, wherein the immune cell secretes one or more polypeptide effector molecules.

Embodiment 3: The immune cell of embodiment 1 or embodiment 2, wherein the label domain comprises a polypeptide derived from structural membrane protein or fetoprotein.

Embodiment 4: The immune cell of any of embodiments 1-3, wherein the polypeptide effector molecule comprises an antibody or a binding fragment thereof that specifically binds to one or more immunomodulators.

Embodiment 5: The immune cell of embodiment 4, wherein the antibody is a VHH antibody.

Embodiment 6: The immune cell of embodiment 4, wherein the immunomodulator is PD-1, PD-L1, CTLA4, LAG-3, TIM-3, BTLA, CD3, CD27, CD28, CD40, CD160, 2B4, 4-1BB, GITR, OX40, VEGF, VEGFR, TGFβ, TGFβR, HVEM or LIGHT.

Embodiment 7: The immune cell of any one of embodiments 1-6, wherein the label domain specifically binds to a bispecific polypeptide comprising a label-binding domain comprising a single chain polypeptide and a cell surface protein-binding domain comprising a single chain polypeptide that binds to a cell surface receptor of a cell.

Embodiment 8: An immune cell that comprises a nucleic acid vector comprising
(a) a promoter region effective for transcription in the immune cell;
(b) a polynucleotide encoding an amino acid sequence of the immune cell activator polypeptide comprising a signal transduction domain, a transmembrane domain, and a label domain; and
(c) a terminator region effective for ending transcription in the immune cell.

Embodiment 9: The immune cell of embodiment 8, which further comprises a second nucleic acid vector comprising
(a) a promoter region effective for transcription in the immune cell;
(b) a polynucleotide encoding an amino acid sequence of one or more secreted polypeptide effector molecules;
(c) a terminator region effective for ending transcription in the immune cell.

Embodiment 10: The immune cell of embodiment 8, wherein the nucleic acid vector further comprises a polynucleotide encoding an amino acid sequence of one or more secreted polypeptide effector molecules.

Embodiment 11: The immune cell of any one of embodiments 8-10, wherein the immune cell activator polypeptide further comprises a binding domain of a VI-11-1 antibody or a single chain variable fragment.

Embodiment 12: The immune cell of any one of embodiments 8-11, wherein the immune cell activator polypeptide comprises a chimeric polypeptide comprising (i) a binding domain of a VI-11-1 antibody or a single chain variable fragment and (ii) the label domain.

Embodiment 13: The immune cell of embodiment 12, wherein the chimeric polypeptide is branched.

Embodiment 14: The immune cell of any one of embodiments 8-13, wherein the label domain comprises a polypeptide derived from a fetoprotein.

Embodiment 15: The immune cell of any one of embodiments 8-13, wherein the label domain comprises a structural membrane protein.

Embodiment 16: The immune cell of any one of embodiments 8-15, wherein the signal transduction domain comprises a co-stimulation domain and a T Cell Receptor (TCR) signaling domain.

Embodiment 17: The immune cell of embodiment 16, wherein the co-stimulation domain comprises CD28, ICOS, CD27, 4-1BB, OX40 or CD40L.

Embodiment 18: The immune cell of embodiment 16 or embodiment 17, wherein the TCR signaling domain comprises CD3ζ or CD3ε.

Embodiment 19: The immune cell of any one of embodiments 16-18, wherein the signal transduction domain comprises CD28 and CD3ζ.

Embodiment 20: The immune cell of any one of embodiments 8-19, wherein the transmembrane domain comprises a domain that participates in immune co-stimulatory signaling.

Embodiment 21: The immune cell of any one of embodiments 8-20, wherein the transmembrane domain comprises CD28.

Embodiment 22: The immune cell of embodiment 21, wherein the CD28 comprises an ITAM domain.

Embodiment 23: The immune cell of any one of embodiments 8-18 and 20-22, wherein the CD3ε domain comprises the amino acids YMNM.

Embodiment 24: The immune cell according to any one of embodiments 8-23, wherein at least one nucleic acid vector further comprises PiggyBac Transposase.

Embodiment 25: The immune cell according to any one of embodiments 8-23, wherein at least one nucleic acid vector further comprises transposon Inverted Terminal Repeat sequences.

Embodiment 26: The immune cell of any one of embodiments 8-25, wherein the polypeptide effector molecule comprises an antibody or a binding fragment thereof that specifically binds to one or more immunomodulators.

Embodiment 27: The immune cell of embodiment 26, wherein the antibody is a VHH antibody.

Embodiment 28: The immune cell of embodiment 26 or embodiment 27, wherein the immunomodulator is PD-1, PD-L1, CTLA4, LAG-3, TIM-3, BTLA, CD3, CD27, CD28, CD40, CD160, 2B4, 4-1BB, GITR, OX40, VEGF, VEGFR, TGFβ, TGFβR, HVEM or LIGHT.

Embodiment 28: The immune cell of any one of embodiments 8-25, wherein the polypeptide effector molecule comprises a cytokine.

Embodiment 30: The immune cell of embodiment 29, wherein the cytokine is TGF-β, VEGF, TNF-α, CCR5, CCR7, IL-2, IL-7, IL-15 or IL-17.

Embodiment 31: The immune cell of any of embodiments 8-30, which is T cell, tumor infiltrating lymphocyte, cytokine activated killer cell, dendritic cell-cytokine activated killer cell, γδ-T cell, natural killer T cell, or natural killer cell.

Embodiment 32: An immune cell activator polypeptide comprising:
(a) a label domain;
(b) a transmembrane domain; and
(c) a signal transduction domain.

Embodiment 33: The immune cell activator polypeptide of embodiment 32, wherein the signal transduction domain comprises a co-stimulation domain and a T Cell Receptor (TCR) signaling domain.

Embodiment 34: The immune cell activator polypeptide of embodiment 33, wherein the co-stimulation domain comprises CD28, ICOS, CD27, 4-1BB, OX40 or CD40L.

Embodiment 35: The immune cell activator polypeptide of embodiment 33, wherein the TCR signaling domain comprises CD3ζ or CD3ε.

Embodiment 36: The immune cell activator polypeptide of embodiment 33, wherein the signal transduction domain comprises CD28 which is linked at its C-terminal end to the N-terminal end of a CD3ε signaling domain.

Embodiment 37: The immune cell activator polypeptide of embodiment 33, wherein the signal transduction domain comprises a co-stimulation domain 4-1BB which is linked at its C-terminal end to the N-terminal end of a CD3ε signaling domain.

Embodiment 38: The immune cell activator polypeptide of any one of embodiments 32-37, wherein the label domain comprises a polypeptide derived from a fetoprotein.

Embodiment 39: The immune cell activator polypeptide of any one of embodiments 32-37, wherein the label domain comprises a structural membrane protein.

Embodiment 40: The immune cell activator polypeptide of any one of embodiments 32-39, wherein the transmembrane domain comprises a domain that participates in immune co-stimulatory signaling.

Embodiment 41: The immune cell activator polypeptide of any one of embodiments 32-40, wherein the transmembrane domain comprises CD28 or a structural membrane protein.

Embodiment 42: The immune cell activator polypeptide of any one of embodiments 32-41, wherein the CD28 comprises an ITAM domain.

Embodiment 43: The immune cell activator polypeptide of any one of embodiments 32-42, wherein the CD3ε domain comprises amino acids YMNM.

Embodiment 44: A nucleic acid vector comprising
(a) a promoter region effective for transcription in an immune cell;
(b) a polynucleotide encoding an amino acid sequence of the immune cell activator polypeptide; and
(c) a terminator region effective for ending transcription in an immune cell.

Embodiment 45: The nucleic acid vector according to embodiment 44, which further comprises transposon Inverted Terminal Repeat sequences.

Embodiment 46: A nucleic acid vector comprising:
(a) a promoter region effective for transcription in an immune cell;
(b) a polynucleotide encoding an amino acid sequence of one or more secreted polypeptide effector molecules.
(c) a terminator region effective for ending transcription in an immune cell.

Embodiment 47: The nucleic acid vector according to embodiment 46, which further comprises transposon Inverted Terminal Repeat sequences.

Embodiment 48: The nucleic acid vector according to embodiment 46 or embodiment 47, wherein the polypeptide effector molecule comprises an antibody or a binding fragment thereof that specifically binds to one or more immunomodulators.

Embodiment 49: The nucleic acid vector according to embodiment 48, wherein the antibody is a VHH antibody.

Embodiment 50: The nucleic acid vector according to embodiment 46 or embodiment 47, wherein the polypeptide effector molecule comprises a cytokine.

Embodiment 51: A bispecific polypeptide comprising:
(a) a label-binding domain (L-bd) comprising a single chain polypeptide domain that specifically binds to the label domain of the immune cell activator polypeptide of any one of embodiments 32-40; and
(b) a cell surface protein-binding domain (CSP-bd) comprising a single chain polypeptide domain that binds to a cell surface receptor of a cell.

Embodiment 52: The bispecific polypeptide of embodiment 51, in which the label-binding domain comprises VHH domain of a camelid IgG.

Embodiment 53: The bispecific polypeptide of embodiment 51 or embodiment 52, which comprises about 15-20 amino acids of a CDR3 domain.

Embodiment 54: The bispecific polypeptide of any one of embodiments 51-53, wherein the cell is a lymphocyte.

Embodiment 55: The bispecific polypeptide of embodiment 54, wherein the lymphocyte is a B cell.

Embodiment 56: The bispecific polypeptide of any one of embodiments 51-53, wherein the cell is a tumor cell.

Embodiment 57: The bispecific polypeptide of embodiment 56, wherein the tumor is lymphoma, non-small cell lung cancer, breast cancer, ovarian cancer, liver cancer, or mesothelioma.

Embodiment 58: The bispecific polypeptide of embodiment 56 or 57, wherein the cell surface protein is EGFR.

Embodiment 59: The bispecific polypeptide of embodiment 56 or 57, wherein the cell surface protein is GPC3.

Embodiment 60: The bispecific polypeptide of any one of embodiments 51-57, wherein the cell surface protein-binding domain specifically binds to EGFR protein expressed on the surface of a tumor cell.

Embodiment 61: The bispecific polypeptide of any one of embodiments 51-57, wherein the cell surface protein-binding domain specifically binds to CD19, CD20 or CD22 on the surface of a lymphoma cell.

Embodiment 62: The bispecific polypeptide of any one of embodiments 51-57, which comprises a VI-11-1 antibody.

Embodiment 63: The bispecific polypeptide of any one of embodiments 51-62, which further comprises one or more domains that provide additional biochemical activities or biological functions.

Embodiment 64: The bispecific polypeptide of embodiment 63, wherein the additional biochemical activities or biological functions comprise: specific binding of a fluorophore, extending the half-life of the bispecific polypeptide in vivo, increasing the affinity of the bispecific polypeptide, and modulating an immune response mediated by a Fc domain.

Embodiment 65: The bispecific polypeptide of any of embodiments 51-62, which comprises additional cell surface protein-binding domain(s) comprising a single chain polypeptide domain(s) that bind to different cell surface receptor(s) of the same or different cell.

Embodiment 66: A kit for in situ production of one or more polypeptide effector molecules proximal to a target cell comprising:
(a) The immune cell according to any one of embodiments 8-31; and
(b) the bispecific polypeptide according to any one of embodiments 51-66.

Embodiment 67: The kit of embodiment 66, wherein the cell surface protein-binding domain specifically binds to CD19 on a B cell.

Embodiment 68: The kit of embodiment 66 or embodiment 68, wherein the cell surface protein-binding domain specifically binds to EGFR, mesothelin, BCMA, MUC1 or GPC3 on a tumor cell.

Embodiment 69: A method for modulating the immune system environment in the locality of a tumor cell in a subject comprising:
(a) administering an effective amount of the immune cell of any one of embodiments 9-31 and an effective amount of a first bispecific polypeptide according to any one of embodiments 51-65 concurrently or sequentially to the subject, wherein the bispecific polypeptide comprises a cell surface protein-binding domain that specifically binds to a cell surface protein of a lymphocyte; and
(b) administering to the subject an effective amount of a second bispecific polypeptide according to any one of embodiments 51-65, wherein the bispecific polypeptide comprises a cell surface protein-binding domain that specifically binds to a cell surface protein of the tumor cell.

Embodiment 70: The method of embodiment 69, further comprising a step performed between steps a. and b. of measuring the amount of the immune cells in the subject.

Embodiment 71: The method of embodiment 70, in which the amount of the immune cells in the blood of the subject is measured.

Embodiment 72: The method of embodiment 70, in which the amount of the immune cells infiltrating the tumor of the subject is measured.

Embodiment 73: The method of any one of embodiments 69-72, wherein the immune cell is T cell, tumor infiltrating lymphocyte, cytokine activated killer cell, dendritic cell-cytokine activated killer cell, γδ-T cell, natural killer T cell, or natural killer cell.

Embodiment 74: The method of any one of embodiments 69-73, wherein the cell surface protein of the lymphocyte is CD19 of a B cell.

Embodiment 75: The method of any one of embodiments 69-74, wherein the tumor cell is lymphoma cell, mesothelial cell, non-small cell lung cancer cell, ovarian cell, liver cancer, or breast cancer cell.

Embodiment 76: The method of embodiment 75, wherein the cell surface protein is EGFR, mesothelin, BCMA, MUC1 or GPC3.

Embodiment 77: A method for modulating the immune system environment in the locality of a tumor cell in a subject comprising:
(a) proliferating a transformed immune cell of the subject in vitro, wherein the immune cell comprises a first nucleic acid vector that comprises a nucleic acid vector comprising
   (i) a promoter region effective for transcription in an immune cell;
   (ii) a polynucleotide encoding an amino acid sequence of an immune cell activator polypeptide; and
   (iii) a terminator region effective for ending transcription in an immune cell;
and comprises a second nucleic acid vector that comprises
   (iv) a promoter region effective for transcription in an immune cell;
   (v) a polynucleotide encoding an amino acid sequence of one or more secreted polypeptide effector molecules; and
   (vi) a terminator region effective for ending transcription in an immune cell;
to obtain proliferated T-cells; and administering the proliferated T-cells into the subject; and
(c) administering to the subject an amount effective to activate the proliferated immune cells to express the immunomodulatory polypeptide of a bispecific polypeptide that comprises a label-binding domain of a determined amino acid sequence that specifically binds to the label domain expressed by the proliferated immune cells and a cell surface protein-binding domain that specifically binds to a cell surface receptor of the tumor cell.

Embodiment 78: The method of embodiment 77, wherein the tumor cell is a mesothelial cell that overexpresses mesothelin and PDL1, and the cell surface protein is mesothelin expressed on the surface of a mesothelial cell, and wherein the effector molecule comprises a VI-11-1 domain that specifically binds to PD-1 or to CD40.

Embodiment 79: The method of embodiment 77 or embodiment 78, wherein the tumor cell is a B cell and the cell surface protein is CD19, CD20 or CD22 on the surface of B cells.

Embodiment 80: The method of any one of embodiments 77-79, wherein the immune cell is T cell, tumor infiltrating lymphocyte cytokine activated killer cell, dendritic cell-cytokine activated killer cell, γδ-T cell, natural killer T cell, or natural killer cell.

1. An immune cell that comprises an expressed immune cell activator polypeptide comprising an intracellular signal transduction domain, a transmembrane domain, and an extracellular label domain, wherein the immune cell secretes one or more polypeptide effector molecules.

2. An immune cell that comprises an expressed immune cell activator polypeptide comprising an intracellular signal transduction domain, a transmembrane domain, and an extracellular chimeric polypeptide comprising a binding domain of a VHH antibody or a single chain variable fragment and a label domain, wherein the immune cell secretes one or more polypeptide effector molecules.

3. The immune cell of claim 1, wherein the label domain comprises a polypeptide derived from structural membrane protein or fetoprotein.

4. The immune cell of claim 1, wherein the polypeptide effector molecule comprises an antibody or a binding fragment thereof that specifically binds to one or more immunomodulators.

5. The immune cell of claim 4, wherein the antibody is a VHH antibody.

6. The immune cell of claim 4, wherein the immunomodulator is PD-1, PD-L1, CTLA4, LAG-3, TIM-3, BTLA, CD3, CD27, CD28, CD40, CD160, 2B4, 4-1BB, GITR, OX40, VEGF, VEGFR, TGFβ, TGFβR, HVEM or LIGHT.

7. The immune cell of claim 1, wherein the label domain specifically binds to a bispecific polypeptide comprising a label-binding domain comprising a single chain polypeptide and a cell surface protein-binding domain comprising a single chain polypeptide that binds to a cell surface receptor of a cell.

8. An immune cell that comprises a nucleic acid vector comprising
(a) a promoter region effective for transcription in the immune cell;
(b) a polynucleotide encoding an amino acid sequence of the immune cell activator polypeptide comprising a signal transduction domain, a transmembrane domain, and a label domain; and
(c) a terminator region effective for ending transcription in the immune cell.

9. The immune cell of claim 8, which further comprises a second nucleic acid vector comprising
(a) a promoter region effective for transcription in the immune cell;
(b) a polynucleotide encoding an amino acid sequence of one or more secreted polypeptide effector molecules;
(c) a terminator region effective for ending transcription in the immune cell.

10. The immune cell of claim 8, wherein the nucleic acid vector further comprises a polynucleotide encoding an amino acid sequence of one or more secreted polypeptide effector molecules.

11. The immune cell of claim 8, wherein the immune cell activator polypeptide further comprises a binding domain of a VHH antibody or a single chain variable fragment.

12. The immune cell of claim 8, wherein the immune cell activator polypeptide comprises a chimeric polypeptide comprising (i) a binding domain of a VHH antibody or a single chain variable fragment and (ii) the label domain.

13. (canceled)

14. The immune cell of claim 8, wherein the label domain comprises a polypeptide derived from a fetoprotein or a structural membrane protein.

15-25. (canceled)

26. The immune cell of claim 8, wherein the polypeptide effector molecule comprises an antibody or a binding fragment thereof that specifically binds to one or more immunomodulators or a cytokine.

27. The immune cell of claim 26, wherein the antibody is a antibody.

28. The immune cell of claim 26, Wherein the immunomodulator is PD-1, PD-L1, CTLA4, LAG-3, TIM-3, BTLA, CD3, CD27,CD28, CD40, CD160, 2B4, 4-1BB, GITR, OX40, VEGF, VEGFR, TGFβ, TGFβR, HVEM or LIGHT.

29. (canceled)

30. The immune cell of claim 26, wherein the cytokine is TGF-β, VEGF, TNF-α, CCR5, CCR7, IL-2, IL-7, IL-15 or IL-17.

31. The immune cell of claim 8, which is T cell, tumor infiltrating lymphocyte, cytokine activated killer cell, dendritic cytokine activated killer cell, γδ-T cell, natural killer T cell, or natural killer cell.

32. An immune cell activator polypeptide comprising:
(a) a label domain;
(b) a transmembrane domain; and
(c) a signal transduction domain.

33-37. (canceled)

38. The immune cell activator polypeptide of claim 32, wherein the label domain comprises a polypeptide derived from a fetoprotein or a structural membrane protein.

39-50. (canceled)

51. A bispecific polypeptide comprising:
(a) a label-binding domain (L-bd) comprising a single chain polypeptide domain that specifically hinds to the label domain of the immune cell activator polypeptide of claim 32; and
(b) a cell surface protein-binding domain (CSP-bd) comprising a single chain polypeptide domain that binds to a cell surface receptor of a cell.

52. The bispecific polypeptide of claim 51, in which the label-binding domain comprises VHH domain of a camelid IgG.

53. The bispecific polypeptide of claim 51, which comprises about 15-20 amino acids of a CDR3 domain.

54. The bispecific polypeptide of claim 51, wherein the cell is a lymphocyte or a tumor cell.

55-56. (canceled)

57. The bispecific polypeptide of claim 54, wherein the tumor is lymphoma, non-small cell lung cancer, breast cancer, ovarian cancer, liver cancer, or mesothelioma.

58. The bispecific polypeptide of claim 54, wherein the cell surface protein is EGFR or GPC3.

59. (canceled)

60. The bispecific polypeptide of claim 54, wherein the cell surface protein-binding domain specifically binds to CD19, CD20 or CD22 on the surface of a lymphoma cell or EGFR protein expressed on the surface of a tumor cell.

61. (canceled)

62. The bispecific polypeptide of claim 51, which comprises a VHH antibody.

63. The bispecific polypeptide of claim 51, which further comprises one or more domains that provide additional biochemical activities or biological functions.

64. The bispecific polypeptide of claim 63, wherein the additional biochemical activities or biological functions comprise: specific binding of a fluorophore, extending the half-life of the bispecific polypeptide in vivo, increasing the affinity of the bispecific polypeptide, and modulating an immune response mediated by a Fc domain.

65. The bispecific polypeptide of claim 51, which comprises additional cell surface protein-binding domains) comprising a single chain polypeptide domain(s) that bind to different cell surface receptor(s) of the same or different cell.

66-68. (canceled)

69. A method for modulating the immune system environment in the locality of a tumor cell in a subject comprising:
(a) administering an effective amount of the immune cell of claim 9 and an effective amount of a first bispecific polypeptide concurrently or sequentially to the subject, wherein the bispecific polypeptide comprises a label-binding domain comprising a single chain polypeptide domain that specifically binds to the label domain of the immune cell activator polypeptide of the immune cell of claim 9 and a cell surface protein-binding domain comprising a single chain polypeptide domain that specifically hinds to a cell surface protein of a lymphocyte; and
(b) administering to the subject an effective amount of a second bispecific polypeptide, wherein the bispecific polypeptide comprises a label-binding domain comprising a single chain polypeptide domain that specifically binds to the label domain of the immune cell activator polypeptide of the immune cell of claim 9 and a cell surface protein-binding domain comprising a single chain polypeptide domain that specifically binds to a cell surface protein of the tumor cell.

70. The method of claim 69, further comprising a step performed between steps a. and b. of measuring the amount of the immune cells in the subject.

71-72. (canceled)

73. The method of claim 69, wherein the immune cell is T cell, tumor infiltrating, lymphocyte, cytokine activated killer cell, dendritic cell-cytokine activated killer cell, γδ-T cell, natural killer T cell, or natural killer cell.

74. The method of claim 69, wherein the cell surface protein of the lymphocyte is CD19 of a B cell.

75. The method of claim 69, wherein the tumor cell is lymphoma cell, mesothelial non-small cell lung cancer cell, ovarian cell, liver cancer, or breast cancer cell.

76. The method of claim 75, wherein the cell surface protein of the tumor cell is EGFR, mesothelin, BCMA, MUC1 or GPC3.

77. A method for modulating the immune system environment in the locality of a tumor cell in a subject comprising:
(a) proliferating a transformed immune cell of the subject in vitro, wherein the immune cell comprises a first nucleic acid vector that comprises a nucleic acid vector comprising
(i) a promoter region effective for transcription in an immune cell;
(ii) a polynucleotide encoding an amino acid sequence of an immune cell activator polypeptide; and
(iii) a terminator region effective for ending transcription in an immune cell; and comprises a second nucleic acid vector that comprises
(iv) a promoter region effective for transcription in an immune cell;
(v) a polynucleotide encoding an amino acid sequence of one or more secreted polypeptide effector molecules; and
(vi) a terminator region effective for ending transcription in an immune cell; to obtain proliferated T-cells; and administering the proliferated T-cells into the subject; and
(b) administering to the subject an amount effective to activate the proliferated, immune cells to express the immunomodulatory polypeptide of a bispecific polypeptide that comprises a label-binding domain of a determined amino acid sequence that specifically binds to the label domain expressed by the proliferated immune cells and a cell surface protein-binding domain that specifically binds to a cell surface receptor of the tumor cell.

78-80. (canceled)


SEQUENCE LISTING (45 sequences)

SEQ ID NO: 1 - protein, 19 aa, artificial
  human signal peptide
MKHLWFFLLLVAAPRWVLS

SEQ ID NO: 2 - protein, 19 aa, artificial
  human signal peptide
MTRLTVLALLAGLLASSRA

SEQ ID NO: 3 - protein, 123 aa, vicugna pacos
QVQLVESGGGLVQAGGSLRLSCAASGDTSFISAAGWYRQAPGKERELVAAITNTGITYYPDSVKGRFTISRDNAKNTVYLQMNNLKPEDTAVYYCNAGAPPPGGLGYDESDYWGQGTQVTVSS

SEQ ID NO: 4 - protein, 4 aa, human
YMNM

SEQ ID NO: 5 - protein, 60 aa, human
MAGHLASDFAFSPPPGGGGDGPGGPEPGWVDPRTWLSFQGPPGGPGIGPGVGPGSEVWGI

SEQ ID NO: 6 - protein, 53 aa, human
MYNMMETELKPPGPQQTSGGGGGNSTAAAAGGNQKNSPDRVKRPMNAFMVWSR

SEQ ID NO: 7 - protein, 54 aa, human
MALSEPILPSFSTFASPCRERGLQERWPRAEPESGGTDDDLNSVLDFILSMGLD

SEQ ID NO: 8 - protein, 38 aa, artificial
  camelid ICAP label domain
MAGHLASDFAFSPPPGGGGDGPGGPEPGWVDPRTWLSF

SEQ ID NO: 9 - protein, 64 aa, human
EVEKTACPSGKKAREIDESLIFYKKWELEACVDAALLATQMDRVNAIPFTYEQLDVLKHKLDEL

SEQ ID NO: 10 - protein, 96 aa, human
SLETLKALLEVNKGHEMSPQVATLIDRFVKGRGQLDKDTLDTLTAFYPGYLCSLSPEELSSVPPSSIWAVRPQDLDTCDPRQLDVLYPKARLAFQN

SEQ ID NO: 11 - protein, 101 aa, human
CSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKL

SEQ ID NO: 12 - protein, 6 aa, artificial
  metalloproteinase cleavable linker
RVLAEA

SEQ ID NO: 13 - protein, 10 aa, artificial
  metalloproteinase cleavable linker
EDVVCCSMSY

SEQ ID NO: 14 - protein, 8 aa, artificial
  metalloproteinase cleavable linker
GGIEGRGS

SEQ ID NO: 15 - protein, 17 aa, artificial
  Factor IXa/Factor VIIa cleavable linker
VSQTSKLTRAETVFPDV

SEQ ID NO: 16 - protein, 254 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSGGGGSGGGGSQVQLVESGGGLVQPGGSLNLSCAASGFDFSSVTMSWHRQSPGKERETVAVISNIGNRNVGSSVRGRFTISRDNKKQTVHLQMDNLKPEDTGIYRCKAWGLDLWGPGTQVTVSS

SEQ ID NO: 17 - protein, 54 aa, human
MLQMAGQCSQNEYFDSLLHACIPCQLRCSSNTPPLTCQRYCNASVTNSVKGTNA

SEQ ID NO: 18 - protein, 23 aa, human
MLQMAGQCSQNEYFDSLLHACIP

SEQ ID NO: 19 - DNA, 624 bp, human
tccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcg

SEQ ID NO: 20 - protein, 208 aa, human
SLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALS

SEQ ID NO: 21 - DNA, 1293 bp, artificial
  M-ICAP CAR ORF encoding DNA
atggccttgccaacggctcgacccctgttggggtcctgtgggacccccgccctcggcagcctcctgttcctgctcttcagcctcggatgggtgcagccccaccaccaccatcaccacggaggaggcggatcttccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcgatctacatctgggcgcccctggccgggacttgtggggtccttctcctgtcactggttatcaccctttactgcaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 22 - protein, 430 aa, artificial
  M-ICAP CAR ORF protein
MALPTARPLLGSCGTPALGSLLFLLFSLGWVQPHHHHHHGGGGSSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1251 bp, artificial
  M-ICAP-SP3 CAR ORF-encoding DNA
atgaagcacctctggttcttcctcctgctggtggcagctcctagatgggtgctgtctcaccaccaccatcaccacggaggaggcggatcttccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcgatctacatctgggcgcccctggccgggacttgtggggtccttctcctgtcactggttatcaccctttactgcaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 24 - protein, 416 aa, artificial
  M-ICAP-SP3 CAR protein
MKHLWFFLLLVAAPRWVLSHHHHHHGGGGSSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 1251 bp, artificial
  [00245] M-ICAP-SP5 CAR ORF-encoding  DNA
atgaccaggctgacagtgctggctctgctggccggactgctggcttcttctagagctcaccaccaccatcaccacggaggaggcggatcttccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcgatctacatctgggcgcccctggccgggacttgtggggtccttctcctgtcactggttatcaccctttactgcaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 26 - protein, 416 aa, artificial
  M-ICAP-SP5 CAR ORF protein
MTRLTVLALLAGLLASSRAHHHHHHGGGGSSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 378 bp, artificial
  M(2339VHH) DNA sequence
cagctgcagctgggcgcctctggcggcggcctggtccagcctggcggctctctgagactgagctgtgccctgtctggcttcacactgagagagctggacgagttcgccatcggctggttcaggcaggcccctggcaaggagagagagggcgtgagctgtatcagcggcacaggcggcatcacacattatgctgacagcgtgaagggcaggttcacaatcagcagagacatcgccaagacaaccgtgtacctgcagatgaatagcctgaacagcgaagacacagccgtgtactactgtgccgccgacgagagatgtacagacagactgatcagacctcctacatattggggacaaggcacccaggtgacagtctcttct

SEQ ID NO: 28 - protein, 126 aa, artificial
  M(2339VHH) protein sequence
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSS

SEQ ID NO: 29 - protein, 120 aa, artificial
  [00249] TCP001-C MC001C BCMA B029(VH) sequence
QVQLVESGGGLVQPGGSLRLSCAASGSITSIYAIGWYRQAPGKLRELVAAITTSGNTFYRDSVKGRFTISRDNAKNTVSLQMNSLKSEDTAVYDCNGAPWGDHAPLVVSWDQGTQVTVSS

SEQ ID NO: 30 - protein, 244 aa, artificial
  TCP011-P CD19 scFv sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 31 - protein, 113 aa, artificial
  TCP021-P EGFR E454(VHH)
QVQLVESGGGLVQPGGSLNLSCAASGFDFSSVTMSWHRQSPGKERETVAVISNIGNRNVGSSVRGRFTISRDNKKQTVHLQMDNLKPEDTGIYRCKAWGLDLWGPGTQVTVSS

SEQ ID NO: 32 - protein, 115 aa, artificial [sequence omitted]

SEQ ID NO: 33 - protein, 247 aa, artificial
  Anti-TGFB scF
QVQLVQSGAEVKKPGASVKVSCKASGYTFTSEWMNWVRQAPGQGLEWMGQIFPALGSTNYNEMYEGRVTMTTDTSTSTAYMELRSLRSDDTAVYYCARGIGNYALDAMDYWGQGTLVTVSSGGGGSGGGGSGGGGSDIQMTQSPSSLSASVGDRVTITCRASESVDFYGNSFMHWYQQKPGKAPKLLIYLASNLESGVPSRFSGSGSGTDFTLTISSLQPEDFATYYCQQNIEDPLTFGGGTKVEIK

SEQ ID NO: 34 - protein, 128 aa, vicugna pacos
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSS

SEQ ID NO: 35 - protein, 119 aa, vicugna pacos
QVQVVESGGGFVQAGGSLRLSCAASTPIISIAYMGWYRQISEKERQLVATINSGGKTYYADSVKGRFTISRDNAKNTLYLQMNMLKPEDTGMYYCAASNKDYNDYDPDWGQGTQVTVSS

SEQ ID NO: 36 - protein, 246 aa, vicugna pacos
DIVLTQSPASLAMSLGERATISCRASESVSVIGAHLIHWYQQKPGQPPKLLIYLASNLETGVPARFSGSGSGTDFTLTISRVQAEDAAIYSCLQSRIFPRTFGQGTKLEIKGSTSGSGKPGSGEGSTKGQVQLVQSGSELKKPGESVKISCKASGYTFTDYSINWVKQAPGQGLKWMGWINTETREPAYAYDFRGRFVFSLDTSASTAYLQISSLKAEDTAVYFCALDYSYAMDYWGQGTLVTVSS

SEQ ID NO: 37 - protein, 287 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSGGGGSGGGGSQVQLVESGGGLVQPGGSLRLSCAASGSITSIYAIGWYRQAPGKLRELVAAITTSGNTFYRDSVKGRFTISRDNAKNTVSLQMNSLKSEDTAVYDCNGAPWGDHAPLVVSWDQGTQVTVSSGGGGSEQKLISEEDLGGGGSHHHHHH

SEQ ID NO: 38 - protein, 413 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSGGGGSGGGGSDIVLTQSPASLAMSLGERATISCRASESVSVIGAHLIHWYQQKPGQPPKLLIYLASNLETGVPARFSGSGSGTDFTLTISRVQAEDAAIYSCLQSRIFPRTFGQGTKLEIKGSTSGSGKPGSGEGSTKGQVQLVQSGSELKKPGESVKISCKASGYTFTDYSINWVKQAPGQGLKWMGWINTETREPAYAYDFRGRFVFSLDTSASTAYLQISSLKAEDTAVYFCALDYSYAMDYWGQGTLVTVSSGGGGSEQKLISEEDLGGGGSHHHHHH

SEQ ID NO: 39 - protein, 282 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSGGGGSGGGGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSEQKLISEEDLGGGGSHHHHHH

SEQ ID NO: 40 - protein, 411 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSEQKLISEEDLGGGGSHHHHHH

SEQ ID NO: 41 - protein, 280 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSGGGGSGGGGSQVQLVESGGGLVQPGGSLNLSCAASGFDFSSVTMSWHRQSPGKERETVAVISNIGNRNVGSSVRGRFTISRDNKKQTVHLQMDNLKPEDTGIYRCKAWGLDLWGPGTQVTVSSGGGGSEQKLISEEDLGGGGSHHHHHH

SEQ ID NO: 42 - protein, 498 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSAAAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVQLVESGGGLVQPGGSLRLSCAASGSITSIYAIGWYRQAPGKLRELVAAITTSGNTFYRDSVKGRFTISRDNAKNTVSLQMNSLKSEDTAVYDCNGAPWGDHAPLVVSWDQGTQVTVSS

SEQ ID NO: 43 - protein, 402 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSAAAGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVQLVESGGGLVQPGGSLRLSCAASGSITSIYAIGWYRQAPGKLRELVAAITTSGNTFYRDSVKGRFTISRDNAKNTVSLQMNSLKSEDTAVYDCNGAPWGDHAPLVVSWDQGTQVTVSSGGGGSEQKLISEEDLGGGGSHHHHHH

SEQ ID NO: 44 - protein, 146 aa, vicugna pacos
QVQLVESGGGLVQPGGSLRLSCAASGSITSIYAIGWYRQAPGKLRELVAAITTSGNTFYRDSVKGRFTISRDNAKNTVSLQMNSLKSEDTAVYDCNGAPWGDHAPLVVSWDQGTQVTVSSGGGGSEQKLISEEDLGGGGSHHHHHH

SEQ ID NO: 45 - protein, 152 aa, vicugna pacos
QLQLGASGGGLVQPGGSLRLSCALSGFTLRELDEFAIGWFRQAPGKEREGVSCISGTGGITHYADSVKGRFTISRDIAKTTVYLQMNSLNSEDTAVYYCAADERCTDRLIRPPTYWGQGTQVTVSSGGGGSEQKLISEEDLGGGGSHHHHHH